(19) United States
(12) Patent Application Publication
(10) Pub. No.: US 2005/0042612 A1
(43) Pub. Date: Feb. 24, 2005

(54) Graft polymer martrices

(76) Inventors: Michael Anthony Hubbard, Avon, NY (US); Scott F. Rosebrough, Avon, NY (US); George L. Oltean, Rochester, NY (US)

Correspondence Address: VENABLE, BAETJER, HOWARD AND CIVILETTI, LLP, P.O. BOX 34385, WASHINGTON, DC, 20043-9998, US

(21) Appl. No.: 10/485,298

(22) PCT Filed: Jul. 30, 2002
(86) PCT No.: PCT/US02/24018

Related U.S. Application Data
(60) Provisional application No. 60/308,119, filed on Jul. 30, 2001.

(51) Int. Cl. C12Q 1/68; G01N 33/53

(52) U.S. Cl. 435/6; 435/7.1; 525/54.1; 525/54.2

(57) ABSTRACT

A three-dimensional, non-crosslinked, linear or branched graft polymer matrix suitable for microassays comprises one or more active chemical moieties having inherent specificity for binding to a chemical, biochemical, or biological probe or target, the moieties being permanently attached to and distributed throughout the graft polymer matrix, optionally including one or more probes.

### FIELD OF THE INVENTION

This invention relates to compositions that can be used to modify or enhance biomaterial and/or device surfaces. Additionally, the invention relates to products having surfaces that are capable of useful bioactive interactions and functions.

### BACKGROUND OF THE INVENTION

An important development in medicine has been the ability to quickly and reliably screen individuals for diseases and more recently genetic markers which may pre-dispose individuals to develop illnesses during their lifetime. The development of clinical diagnostic methods based on gene expression profiles is growing rapidly due to genetic information gathered by the Human Genome Project, the generation of animal models to study human disease and many other genomic and proteomic approaches being applied to decipher the molecular pathogenesis of disease over the last decade.

A consequence of these efforts has been to strive for the development of high speed, high throughput, and highly reproducible DNA and protein microarray technologies. The basic concepts of these technologies are the same as those for enzyme based immunoassay and RNA-based northern blotting techniques that have been used for years to detect protein antigens and gene expression levels. Highly specific probes generated to query a large number of molecules (e.g. antigens or nucleic acid sequences) are attached to the surface of a sample slide and exposed to target molecules generated from a wide variety of biological materials. If target molecules are present in the hybridization cocktail, they will bind with high affinity and specificity to substrate-bound probe molecules immobilized on the slide. The slide is processed and imaged in a way that only areas containing bound target/probe substrate are detectable and fully quantifiable through radiolabeling, enzymatic colorimetry, fluorescence spectroscopy, mass spectroscopy or other techniques known to those skilled in the art.

An important difference between gene expression approaches using microarrays and more standard molecular biological approaches is their relative size scales. An example of this is the comparison of a microtiter plate based approach where the well of a typical 96-well microtiter plate is about 1 cm in diameter and can contain up to several hundred microliters of probe-containing solution. In contrast, a DNA microarray spot is only about 25-100 μm in diameter and can be printed with just 200-500 pl of probe-containing solution. Thus, a substrate the size of a microscope slide can contain an array of tens of thousands of spots, with each spot containing a different molecular probe or probe concentration. With high resolution and high speed automated printing processes that are now available, microarrays can be manufactured reliably and on a large scale.

One common microarray surface structure comprises a linker molecule connecting the surface of the substrate (typically glass) with one end of the probe molecule. Typical linker molecules for DNA microarrays are amino-terminated silanes. They bind covalently to the glass surface through the silane end and are photo-crosslinked through the amine end to a DNA probe. Alternatively, aldhehyde-terminated linker molecules can be used. In this scheme, the aldehyde functionalities form Schiff base adducts with amine groups conjugated to the thymidine residues of the DNA oligomer. Protein microarray substrates containing binding proteins such as monoclonal antibodies, single chain antibodies and/or peptides, for example, may also be prepared in this manner.

A significant limitation to this approach is the inability to achieve more than one layer of probes on the slide surface. This two-dimensionality imposes an important spatial constraint given the surface area of a microarray spot. The inability to stack probes in three dimensions directly impacts the maximum dynamic range and sensitivity that may be achieved with this substrate structure. A simple calculation underscores the significance of this limitation. The surface density of oligonucleotides on aminated glass surfaces has been estimated at 0.1 pmol/mm² (˜1 molecule per 1600 Å²), meaning that there are ˜1.2×10⁸ sites available for probe binding on a 50 μm diameter spot. A 260 pl drop of a DNA oligomer solution containing 500 ng/μl DNA will contain ˜4×10¹¹ oligomer units (assuming a 20 unit oligomer and average MW of 200/nucleic acid residue). Thus, such a droplet will have over a 1000-fold more genetic material than the surface is capable of accepting. It is clear that the limiting factor in increasing microarray dynamic range is not the concentration of the target in solution but the rather the number of sites on the microarray to which the target can bind. As microarray features become smaller and smaller with the continued interest in miniaturization, the space available for those binding sites will continue to decrease. Construction of microarray surfaces that are capable of accepting probe molecules in three dimensions will allow a substantial increase in the density of probes per unit area on the microarray surface. Three-dimensional surfaces also make it possible for probe or target molecules to attach to the array without steric hindrance or surface interference. This sort of steric hindrance could most likely be associated with the use of large proteins or nucleic acid sequences. It is for this reason that we have developed technology that will allow the three-dimensional placement of probes on microarray surfaces.

One solution to these problems is the development of three-dimensional matrices. Such three-dimensional matrices should be rapidly porous to liquids so that probes may be attached throughout the depth of the matrix, thus making it possible to achieve higher spatial densities of probe molecules than has been achieved previously. Increased matrix porosity will improve the efficiency and degree of the target/probe hybridization process. It is well known that protein interactions are adversely affected due to the inherent instability and denaturation of proteins adhered on solid surfaces. This limits the use of specific binding proteins, for example as found with antibody/antigen and receptor/ligand interactions, on two-dimensional surfaces. Therefore, a three-dimensional matrix will allow for a pseudo-solution phase where polymer-bound and target proteins are removed from the solid surface and evenly dispersed throughout the matrix increase binding efficiency. A number of approaches have been taken to develop three-dimensional surfaces capable of binding probe molecules to them. For example, hydrogel layers in various configurations are often cumbersome and expensive and/or unreliable to manufacture in large volume. Potential problems with some of these approaches arise from the possible limited access to the inner part of the matrix of probes and targets as a result of the crosslinked matrix.

Duran et al. disclose in U.S. Pat. No. 5,858,653 reagent compositions for covalent attachment of target molecules, such as nucleic acids, onto the surface of a substrate. The reagent compositions include groups capable of attracting the target molecule as well as groups capable of covalently binding to the target molecule, once attracted. Optionally, the compositions can contain photoreactive groups for use in attaching the reagent composition to a surface. This method has several limitations and disadvantages. First, it can be used to bond onto polymeric surfaces only. In addition, initiator contained in the aqueous copolymer medium may lead to non-grafted polymer and block copolymer, which requires crosslinking to be permanently retained in the layer. Finally, the patent does not disclose methods and compositions for use on glass surfaces, and therefore does not enable one skilled in the art to make coatings on glass without undue experimentation.

Hahn, et al. disclose in U.S. Pat. No. 6,174,683, methods for preparing a biochip wherein the biomolecular probe to be used with the biochip is alternately bound to a hydrogel prepolymer prior to or simultaneously with polymerization of the prepolymer. In particularly preferred embodiments, a polyurethane-based hydrogel prepolymer is derivitized with an organic solvent soluble biomolecule, such as a peptide nucleic acid probe in aprotic, organic solvent. Following derivitization of the prepolymer, an aqueous solution, for example sodium bicarbonate, preferably buffered to a pH of about 7.2 to about 9.5, is added to the derivatized prepolymer solution to initiate polymerization of the hydrogel. Alternatively, a water soluble biomolecule, such as DNA or other oligonucleotide, is prepared in an aqueous solution and added to the polyurethane-based hydrogel prepolymer such that derivitization and polymerization occur, essentially, simultaneously. While the hydrogel is polymerizing, it is microspotted onto a solid substrate, preferably a silinated glass substrate, to which the hydrogel microdroplet allegedly becomes covalently bound. Most preferably the hydrogel microdroplets are at least about 30 μm thick, for example about 50 μm to about 100 μm thick.

This process is complicated compared to the more usual technique of hybridizing directly onto a hydrogel surface. In addition, it uses organic solvents which may denature nucleotides, the hydrogel microdroplets may not adhere well to glass surfaces, the hydrogel droplets may not be printed in as dense a format as conventional oligo solutions, and the technology is expensive to accomplish because of its complexity.

Chenchik, et al. disclose in U.S. Pat. No. 6,087,102 arrays of polymeric targets stably associated with the surface of a rigid solid support. The polymeric targets are arranged according size via electrophoresis. The polymeric targets are generally biopolymeric compounds, e.g. nucleic acids and proteins, where ribonucleic acids and proteins are the preferred polymeric targets. The technology uses multiple silanization, rinse, and polish steps to first prepare the surface for coating with a hydrogel. The plates must be rinsed and polished between and following each silanization step, a cumbersome and expensive process. The gel polymerization is conducted between plates that are clamped together. This gel is also crosslinked following the polymerization step, which can slow diffusion of aqueous fluids into it.

Lockhart, et al. disclose in U.S. Pat. No. 6,040,138 methods of monitoring the expression levels of a multiplicity of genes. The methods involve hybridizing a nucleic acid sample to a high density array of oligonucleotide probes where the high density array contains complimentary subsequences to target nucleic acids in the nucleic acid sample. In one embodiment, the method involves providing a pool of target nucleic acids comprising RNA transcripts of one or more target genes, or nucleic acids derived from the RNA transcripts, hybridizing said pool of nucleic acids to an array of oligonucleotide probes immobilized on the surface, where the array comprises more than 100 different oligonucleotides and each different oligonucleotide is localized in a predetermined region of the surface, the density of the different oligonucleotides is greater than about 60 different oligonucleotides per cm², and the oligonucleotide probes are complimentary to the RNA transcripts or nucleic acids derived from the RNA transcripts; and quantifying the hybridized nucleic acids in the array. This technology is complicated to manufacture. Although it discloses certain probes and probe arrangements, it does not disclose new substrate technology.

Pirrung et al. disclose in U.S. Pat. No. 6,225,625 a method and apparatus for preparation of a substrate containing a plurality of sequences. Photoremovable groups are attached to a surface of a substrate. Selected regions of the substrate are exposed to light so as to activate the selected areas. A monomer, also containing a photoremovable group, is provided to the substrate to bind at the selected areas. The process is repeated using a variety of monomers such as amino acids until sequences of a desired length are obtained. Detection methods and apparatus are also disclosed. This process is designed to facilitate efficient synthesis of polynucleotides or other biopolymers. However, the process is limited to a two-dimensional configuration.

Felder et al. disclose in U.S. Pat. No. 6,232,066 compositions, apparatus and methods for concurrently performing multiple, high throughput, biological or chemical assays, using repeated arrays of probes. A combination of the invention comprises a surface, which comprises a plurality of test regions, several of which are substantially identical, wherein each of the test regions comprises an array of generic anchor molecules. The anchors are associated with bi-functional linker molecules, each containing a portion which is specific for at least one of the anchors and a portion of which is a probe specific for a target of interest. This technology produces a two-dimensional surface which necessarily limits the amount of probe that can be arrayed in a given area.

Anders et al. disclose in U.S. Pat. No. 6,096,369 a process for making the surface of polymeric substrates hydrophilic, which includes coating the surface of a polymeric substrate with a solution of a macroinitiator, wherein the macroinitiator includes a polymer framework and side chains attached to the polymer framework, and wherein at least one of the side chains includes at least one free-radical-forming group. Optionally, a hydrophilic vinyl monomer or monomers may then be free-radical polymerized or graft polymerized onto the macroinitiator-coated substrate. A crosslinking vinyl monomer may optionally be used together with the macroinitiator or the hydrophilic vinyl monomer. This process requires a polymeric substrate. The hydrophilic polymers are not designed to react with biomaterial probes.

Turner et al. disclose in U.S. Pat. No. 5,948,62 a stamp for transferring molecules and molecular patterns to a substrate face which includes a backing and a polymeric gel bound to the backing and loaded with the molecular species. Where the molecule to be patterned is a biomolecule, such as a protein or nucleic acid, the polymeric gel is typically a hydrogel, such as sugar-based polyacrylates and polyacrylamides. The process includes preparation of silanized glass plates and formation thereon of hydrogel layers via polymerization of 6-acryloyl-B-O-methylgalactoside (2% crosslinking) and N,N′-methylenebisacrylamide. A relief image-wise pattern is created on the hydrogel surface, which is used to transfer monoclonal antibodies or other biomolecules onto a substrate. This process uses a crosslinked hydrogel like a relief printing plate, and is unlikely to achieve the same level of pattern sharpness that is achieved by modem printing methods. In addition, the process is cumbersome to accomplish, and may be expensive.

Jannsen, et al. disclose in U.S. Pat. No. 4,978,481 a process for encapsulating a preformed polymeric substrate by forming peroxide or hydroperoxide sites on the substrate surface using ozone and then carrying out a crosslinked graft polymerization of selected ethyleneically unsaturated monomers both on and surrounding (encapsulating) said substrate. This method relies on the use of ozone which is undesirable. The control of the density of graft polymer links is limited, and no provisions are incorporated for probe linkages.

Clapper, et al. disclose in U.S. Pat. No. 6,121,027 a polyfunctional reagent having a polymeric crosslinked backbone, one or more pendant photoreactive moieties, and two or more pendant bioactive groups. The reagent can be activated to form a coating on a polymeric surface. The pendant bioactive groups function by promoting the attachment of specific molecules or cells to the coated surface. This method is cumbersome and requires crosslinking to entrain non-grafted polymer to sustain layer integrity in aqueous media.

Matsuda et al. disclose in U.S. Pat. No. 5,128,170 a medical device having a biocompatible surface wherein a hydrophilic polymer is bonded onto a surface of the medical device covalently through a nitrogen atom, and a method for manufacturing such a medical device is provided. The process includes the steps of applying a hydrophilic polymer having an azido group and/or a composition comprising a compound having at least two azido groups and a hydrophilic polymer onto the surface of the medical device, and irradiating the biocompatible material with light so that the hydrophilic polymer is bonded to the medical device surface. This process is likely to produce non-grafted polymer or copolymer which must be dealt with in a washing step to remove non-grafted polymer or copolymer from the layer, or crosslinking so that the non-grafted polymer or copolymer is retained permanently in the coated layer. No provision is made to link probes in/on the coated surface.

Surface matrices must have uniform thickness, high diffusivity of unbound target throughout the matrix, be suitable for patterning of probe-rich and probe-poor areas and exhibit negligible non-specific binding. Attachment of graft polymers is a highly useful method of surface modification. The functional utility of a graft polymer-modified surface is in certain cases dependent upon the surface density of grafts. Graft polymer surface coatings are low density and porous by nature when compared to most other polymer coatings, this allows for the defusing and binding of larger molecules throughout the coating. This porosity can be further enhanced if the graft polymer chain density and length can be controlled. Furthermore, all surfaces are not inherently susceptible to the formation of permanent graft polymer attachment.

Thus, there is a need for a three-dimensional non-crosslinked linear or branched graft polymer matrix comprising one or more active chemical moieties having inherent specificity for binding to chemical or biochemical, or biological probes or targets, wherein said moieties are permanently attached to and distributed throughout the graft polymer matrix, with a controlled graft polymer chain density and length. There also is a need for a method for graft polymer surface modification that allows for control of the surface density and chain length of the resulting graft polymer matrix, and also makes it possible to graft polymers onto surfaces on which graft polymerization was not heretofore possible. There also is a need to spatially separate proteins from the proximity of a surface to allow efficient protein binding interactions and to avoid denaturation, inactivation and steric hindrance.

### SUMMARY OF THE INVENTION

The invention provides for a novel three-dimensional, non-crosslinked graft polymer matrix containing one or more chemical, biochemical or biological moieties attached to the graft polymer chain, said moieties having been selected to have reactivity with specific probe or target molecular species.

The graft polymer matrix differs significantly from those generated from other emerging three dimensional coating technologies in that its advantages are achieved without the need for covalent crosslinks. The invention calls for individual polymer chains grafted to a surface, as depicted schematically in FIG. 8. Probe reactive groups (active chemical moieties) can be incorporated into the graft polymer matrix that bind to either DNA or protein based probes. The system is not confined to any particular type of linking or reactive group chemistry. The term “non-crosslinked” is intended to refer to a polymer matrix in which the benefits of a porous, coherent material are achieved with individual polymer chains bound to the substrate without the requirement of extensive crosslinking, and the term “non-crosslinked” is intended to distinguish known crosslinked polymer matrices as in prior publications and products described here and otherwise known.

The system is not limited to, but is well suited for microarray assay and nanotechnology. The graft polymer matrix also can include a spacer arm between the probe reactive groups and graft polymer backbone to reduce steric hindrance from the graft polymer backbone. Moreover, for protein assays the printed probe, e.g. a monoclonal antibody or enzyme, can exist in a pseudo solution phase. It is well know that proteins denature or change conformation after binding to a solid surface (e.g. polystryrene microtiter plates). Using the graft polymer matrix the proteins may be attached by a single endpoint, and are spatially removed from the solid interface and can exist in a semi-soluble state. This may be particularly relevant to discovery research where the goal is to develop therapeutic reagents, such as peptides of monoclonal antibodies, for in vivo targets. In these situations the selection of target-specific agents is only relevant if the target (e.g. antigen) is present in its native in vivo state during screening.

The graft polymer matrix may also contain long pendant side-chains (structural modifiers) that may provide structural integrity to the coatings without the rigidity imposed by covalent crosslinks. The degree of hydrophilicity can be controlled by the structural modifiers or the monomeric groups incorporated into graft polymer matrix. This will be important for fine-tuning the matrix, for example, if one wishes to control the spot diffusion during microarray printing. To increase diffusion into the matrix one may wish to increase the hydrophilic nature of the matrix. This may also increase the performance of assays that require hydrophilic conditions, such as found with typical protein assays. This greater flexibility and the controlled graft polymer chain surface density allows for better probe and target diffusion throughout the matrix compared to other three dimensional systems. In addition, the open structure of graft polymer matrix surfaces will allow easier and more efficient washing, thus reducing nonspecific binding due to entrapment in known densely packed coated or crosslinked polymer chains. By controlling the length of the graft polymer chains and the distribution of probe reactive groups and structural modifiers within each grafted chain one can tailor the assay milieu to meet specific assay or biological performance needs. The structural modifiers may also serve as buffers, or to increase or decrease ionic binding, by the incorporation of appropriate charged groups. A limited amount of crosslinking is also contemplated within the scope of the invention so long as the polymer chains are directly bound to the surface, and the crosslinking does not interfere with the functionality of the matrix. By combining these features, the invention substantially increases the sensitivity and dynamic range of microarrays for both genomics and proteomics.

In one aspect, the invention provides a method for producing a three-dimensional, non-crosslinked graft polymer matrix having chemical moieties permanently attached to it, said moieties being distributed throughout the matrix in a known and controlled manner.

In yet another aspect, the invention provides for an article having at its surface a permanently attached three-dimensional, non-crosslinked graft polymer matrix containing probe molecules, said probe molecules having inherent specificity for binding to target chemical or biochemical species for which information on its presence or concentration in a sample is of interest. The probe molecules may be permanently attached to the graft polymer matrix and their distribution throughout the matrix may be chosen in such a way as to provide optimal sensitivity and dynamic range to the detection of said target molecules. In a preferred embodiment of this aspect of the invention, the probes are attached as side-chains to the backbone of the graft polymer.

In still another aspect, the invention provides a method for permanently attaching a multiplicity of graft polymer chains to a surface wherein the density of graft chains per unit area can be controlled to allow for large probe and target binding throughout the graft polymer matrix leading to increased sensitivity and dynamic range.

In still another aspect, the invention provides a method for permanently attaching a multiplicity of graft polymer chains to a surface wherein the length of graft chains can be controlled to allow for increased sensitivity and dynamic range.

One object of the invention is to provide a three-dimensional, non-crosslinked, linear or branched graft polymer matrix containing one or more active chemical moieties having inherent specificity for binding to chemical or biochemical, or biological probes or targets, wherein said moieties are permanently attached to and distributed throughout the graft polymer matrix. In a preferred embodiment of this aspect of the invention the biological targets are selected from the group consisting of viruses, fungi, parasites, and bacteria. In another preferred embodiment, the chemical or biochemical functional targets are selected from the group consisting of molecules or molecular fragments of DNA, RNA, protein, carbohydrates and lipids. In still other preferred embodiments, the probe or target may be a toxin. In still another preferred embodiment, the active chemical moiety is selected from the group consisting of amines, carboxylic acids, epoxides, aldehydes, sulfhydryls, thioesters, haloacetamides and carboxylic acid succinimidyl esters. In an especially preferred embodiment of this aspect of the invention, the active chemical moiety is the N-hydroxysuccinimidyl ester of a carboxylic acid.

It is another object of the invention to provide a three-dimensional, non-crosslinked, linear or branched graft polymer matrix containing one or more active chemical moieties imparting specific functional utility to the coating matrix, wherein said moieties are permanently attached to and distributed throughout the graft polymer matrix, with a controlled graft polymer chain density and length. In one preferred embodiment of this aspect of the invention, the functional utility is an enhanced ability to be imaged by an imaging method selected from the group consisting of magnetic resonance imaging, computer tomography, x-ray radiology, fluorescence, and ultrasonography. In another preferred embodiment, the functional utility is an enhanced resistance to infection and thrombosis.

It is another object of the invention to provide an article having on its surface a multilayered coating comprising:
a) An adhesive polymer layer bound to the surface of the article
b) A three-dimensional, non-crosslinked, linear or branched graft polymer matrix containing one or more chemical moieties that are permanently attached to and distributed throughout the graft polymer matrix wherein said chemical moieties impart specific functional utility to the article.

In one preferred embodiment of this aspect of the invention, the chemical moieties are attached with side-chain spacer arms to the backbone of the graft polymer. Monomers having functional groups that remain intact during the graft polymerization process appear as attached side chains on the graft polymer backbone.

In one preferred embodiment of this aspect of the invention, the thickness of the matrix coating upon the article can be varied, for example, by varying the time of the graft polymerization reaction. Such variation may be desirable depending upon the number of different probes and targets, to be used, sample size, and other factors dependent upon the type of measurements to be made.

In one preferred embodiment of this aspect of the invention, the article is a microarray. The article according to this aspect of the invention may contain microchannels, one purpose of which is to facilitate the diffusion of large probe and target species to reactive sites within the matrix. The size of these microchannels being determined by the open space between the graft polymer chains or their surface density.

Other preferred embodiments of this aspect of the invention include devices whose performance and utility could be enhanced through the application of this invention. Examples of such devices include multi-welled plates, drug release devices that bind drugs or other therapeutic compounds for in vivo release; devices which sequester and thus remove target compounds from solution, such as in dialysis, or the like or other applications that could be envisioned by a person of skill in the art who would understand how to make the necessary adaptations.

In another preferred embodiment of this aspect of the invention, the article is a chemical sensor. The article according to this aspect of the invention may contain chemical moieties that are permanently attached to the graft polymer matrix. Said chemical moieties may have reactivity towards specific nonbiological chemical species or classes of species. Representative examples of such classes include, but are not limited to, monovalent metal ions, divalent metal ions, transition metal ions, inorganic halides, carbonates, sulfates, phosphates, borates, arsenates, zero valent heavy metals, and the like. Upon attachment of one or more of the chemical species to the graft polymer, one or more physical properties of said coating will be altered in a way that is detectable by an appropriate detection device. Representative examples of physical properties that could be altered are, but are not limited to, electrical conductivity, capacitance or impedence, paramagnetism or diamagnetism, optical clarity, optical transmittance over a narrow or wide range of wavelengths, or the like.

In another preferred embodiment, the article has an enhanced ability to be imaged by an imaging method selected from the list magnetic resonance imaging, computer tomography, x-ray radiology, and ultrasonography relative to analogous articles that are not coated as described herein.

In one particularly preferred embodiment, the functional utility is an enhanced resistance to infection. Coatings of this type are particularly useful on medical devices.

Medical devices that may be coated according to the methods of the invention include catheters (including, for example, arterial, short term central venous, long term tunneled central venous, peripheral venous, peripherally insertable central venous, pulmonary artery Swan-Ganz, PTCA or PTA, and vascular port), dialysis devices, introducers, needles (including, for example, amniocentesis, biopsy, introducer), obdurators, pacemaker leads, penile prosthesis, shunts (including, for example, arteriovenous and hydrocephalus shunts), small or temporary joint replacements, stents (e.g. biliary, coronary, neurological, urological, and vascular), syringes, tubes (e.g. drain, endotracheal, gastroenteric, nasogastric), urinary devices (e.g. long term and tissue bonding), urinary dilators, urinary sphincters, urethral inserts, and wound drains. Other devices that may be advantageously coated will be familiar to those of skill in the art.

The surface of an article to be coated according to methods of the invention may be comprised of glass, metal, or polymeric material. The initiator can generally be applied directly to an article having a polymeric surface without using a primer, whereas in the case of a glass or metal surface, a primer may be necessary or desirable to ensure optimal adhesion of the initiator and the graft polymer matrix to the surface. It is therefore a further object of the invention to provide a primer and a method of applying a three dimensional matrix according to the invention that includes a primer, for use on surfaces where the use of a primer may be necessary or desirable. The primer may comprise a solution of a polymer or mixture of polymers in an organic solvent or mixture of organic solvents. The primer solution is applied to the surface of the article either prior to, or simultaneously with, the initiator. Suitable primer solutions can be prepared, for example, using organic solvents such as tetrahydrofuran, toluene, methylethylketone combined with a suitable polymer or polymer mixture. It has been found that in some instances the initiator may be combined with the primer solution to produce superior results.

It is therefore one object of the invention to provide a method for applying a three-dimensional, non-crosslinked, linear or branched graft polymer matrix with a controlled graft polymer chain surface density to an uncoated surface, said method comprising the steps of:
a) applying to said surface a solution comprising
   i) an organic solvent or mixture of organic solvents,
   ii) a polymer or mixture of polymers which is soluble in said solvent or solvent mixture and
   iii) a radical initiator which is soluble in said solvent or solvent mixture, said initiator being capable of generating reactive radical sites on one or more of the polymers present on the-coated substrate surface and initiating a graft polymerization reaction on said polymer-coated surface by generating reactive radical sites thereon.
b) removing the solvents to leave upon the surface of the substrate a coating of initiator dissolved in polymeric primer
c) immersing the polymer-coated surface in a medium comprising one or more monomers in solution that are capable of reacting with the reactive sites created by surface bound initiator to form a polymer chain grafted onto said polymer-coated surface;
d) initiating a graft polymerization reaction on said polymer-coated surface by generating reactive radical sites thereon,
e) graft polymerizing onto the polymer-coated surface the reactive monomers from the medium by forming covalent bonds between monomer molecules and the polymeric surface at reactive radical sites on the polymer-coated surface.

The graft polymer surface density is controlled by mixing together different ratios of reactive polymers and unreactive polymers and/or by using different concentrations of initiator.

In a preferred embodiment of this aspect of the invention, the monomer solution used comprises at least one monomer that, when incorporated into the three-dimensional non-crosslinked graft polymer matrix, provides the resulting graft polymer with side-chain chemical moieties that are permanently attached to and distributed throughout the graft polymer matrix, said side-chain chemical moieties having inherent specificity for binding to chemical, biochemical, or biological probes or targets. The side-chain chemical moieties may be modified to alter their reactivity. The side chain moieties may have inherent specificity for binding to chemical, biochemical, or biological probes or targets, or have other features that impart specific functional utility to the article.

It is another object of the invention to provide a method for applying a three-dimensional, non-crosslinked, linear or branched graft polymer matrix with a controlled graft polymer chain density to an uncoated surface, said method comprising the steps of:
a) applying to said surface a solution comprising:
   i) an organic solvent or mixture of organic solvents and
   ii) a polymer or mixture of polymers which is soluble in said solvent or solvent mixture
b) removing the solvents to leave a coating of polymeric primer upon the substrate surface,
c) exposing said polymer-coated surface to an initiator capable of generating reactive radical sites on one or more of the polymers present on the coated surface of the substrate and initiating a graft polymerization reaction on said surface by generating reactive radical sites thereon
d) immersing the polymer-coated surface in a medium comprising:
   i) one or more monomers in solution that are capable of reacting with the reactive sites created by surface bound initiator to form a polymer chain grafted onto said surface; and
   ii) a concentration of solute in sufficient concentration to induce a ‘salting-out effect’ wherein reactive monomers preferentially localize at the surface of the substrate, wherein the initiator used is insoluble or poorly soluble;
e) initiating a graft polymerization reaction on said coated surface by generating reactive radical sites thereon,
f) graft polymerizing onto the coated surface the reactive monomers from the medium by forming covalent bonds between monomer molecules and the surface at reactive radical sites on the polymer coated surface.

The graft polymer surface density is controlled by mixing together different ratios of reactive polymers and unreactive polymers and/or by changing the amount of initiator the polymer coated surface is exposed to.

It is another object of the invention to provide a method for applying a three-dimensional, non-crosslinked, linear or branched graft polymer matrix with a controlled graft polymer chain density to an uncoated glass surface, said method comprising the steps of:
a) applying to a glass surface a solution comprising:
   i) an organic solvent or mixture of organic solvents with water and
   ii) a silane monomer or mixture of silane monomers and unreactive silanes which is soluble in said solvent or solvent water mixture
b) removing the solvents to leave a coating of silane attached monomers or a mixture of silane attached monomers and silane attached unreactive groups.
c) exposing said silane coated surface to an initiator capable of generating reactive radical sites on one or more of the silane attached groups present on the coated surface of the substrate and initiating a graft polymerization reaction on said surface by generating reactive radical sites thereon
d) immersing the silane-coated surface in a medium having one or more monomers in solution that are capable of reacting with the reactive sites created by surface bound initiator to form a polymer chain grafted onto said surface;
e) initiating a graft polymerization reaction on said coated surface by generating reactive radical sites thereon,
f) graft polymerizing onto the coated surface the reactive monomers from the medium by forming covalent bonds between monomer molecules and the surface at reactive radical sites on the silane coated surface.

The graft polymer surface density is controlled by mixing together different ratios of reactive silane attached monomers and unreactive silane attached groups on the glass surface and/or by changing the amount of initiator the silane coated surface is exposed to.

It is another object of the invention to provide a method for applying a three-dimensional, non-crosslinked, linear or branched graft polymer matrix with a controlled graft polymer chain density to an uncoated glass surface, said method comprising the steps of:
a) applying to a glass surface a solution comprising:
   i) an organic solvent or mixture of organic solvents with water and
   ii) a silane with an attached initiator or mixture of silane initiators and unreactive silane groups which is soluble in said solvent or solvent water mixture
b) removing the solvents to leave a coating of silane attached initiator or a mixture of silane attached initiators and silane attached unreactive groups.
c) immersing the silane-coated surface in a medium comprising one or more monomers in solution that are capable of reacting with the reactive sites created by surface bound initiator to form a polymer chain grafted onto said surface;
d) initiating a graft polymerization reaction on said coated surface by generating reactive radical sites thereon,
e) graft polymerizing onto the coated surface the reactive monomers from the medium by forming covalent bonds between monomer molecules and the surface at reactive radical sites on the silane coated surface.

The graft polymer surface density is controlled by mixing together different ratios of reactive silane attached initiators and unreactive silane attached groups on the glass surface.

In a preferred embodiment of this aspect of the invention, the monomer solution used comprises at least one monomer that, when incorporated into the three-dimensional non-crosslinked graft polymer matrix, provides the resulting graft polymer with chemical moieties that are permanently attached to and distributed throughout the graft polymer matrix, said chemical moieties having inherent specificity for binding to chemical, biochemical, or biological probes or targets. The chemical moieties may be modified to alter their reactivity. The moieties have inherent specificity for binding to chemical, biochemical, or biological probes or targets, or have other characteristics that impart specific functional utility to the article. In an especially preferred embodiment of this aspect of the invention, the chemical moieties are attached as side-chains to the backbone of the graft polymer matrix and are separated from the graft polymer backbone by a side chain spacer arm that can enhance probe and target binding efficiency.

In a particularly preferred embodiment of the invention, graft polymer matrix is applied to the article surface in a pattern. This facilitates the placement of different types of reactive probes, and the reading of results once the matrix has been allowed to react with a test sample.

The graft polymer matrices of the present invention may be constructed and used so as to provide an increased capacity and efficiency for a single type of reactive group or probe, or may be constructed and used for mixtures of or multiple layers of probes/functional groups in a single location.

Matrices prepared according to the present invention have a surface density that can be controlled as well as controlled chain lengths to provide a uniform surface for printing and hybridization.

It will be appreciated that the individual features and aspects of the invention are intended to be combined in all combinations that will be operable and practical, and that all of such combinations are intended to be included within the invention.

### BRIEF DESCRIPTION OF THE DRAWINGS

FIGS. 1A-1C graph the reaction of a N-hydroxysuccinimide-probe with amine-containing graft polymer surface (calorimetric and fluorescence analysis).

FIGS. 2A and 2B show reaction of an amine-containing probe with a NHS-modified graft polymer modified surface.

FIGS. 3A, 3B show an increase in color density corresponding to increase in printed biocytin concentration across the graft polymer surface. FIG. 3A shows amount of biocytin printed. FIG. 3B shows images after development.

FIG. 4

FIG. 4A Weight gain of glass slides primed with either low or high concentration of initiator in primer

FIG. 4B Amount of amino groups in graft coat on glass slides primed with either low or high concentration of initiator in primer.

FIG. 4C Composite fluorescent image of FITC binding to amino groups in graft coat on glass slides with either low or high concentration of initiator in primer. A false color scale spectrum indicates degree of binding (red highest).

FIG. 4D Confocal microscopy depth measurements of FITC labeled amino groups on graft coat glass slides with either low or high concentration of initiator.

FIG. 5

FIGS. 5A, 5B Macroanalysis of NHS-graft coat with biocytin printing and streptavidin-Cy3 detection.

FIG. 5C Microarray analysis of NHS-graft coat with biocytin printing and streptavidin-CY3 detection

FIG. 5D Microarray analysis of NHS-graft coat printed with NH2-oligos, oligo and cDNA controls.

FIG. 6 shows microarray analysis of SH-graft coat slides printed with acrylite-oligos and control reagents.

FIG. 7 shows primary amine concentrations and graft copolymer chains

FIG. 8 is a schematic depiction of a graft polymer matrix

### DETAILED DESCRIPTION OF THE INVENTION

In describing preferred embodiments of the present invention, specific terminology is employed for the sake of clarity. However, the invention is not intended to be limited to the specific terminology so selected. It is to be understood that each specific element includes all technical equivalents which operate in a similar manner to accomplish a similar purpose. Each reference cited herein is incorporated by reference as if each were individually incorporated by reference.

Definitions
Graft polymer: A linear or branched polymer or copolymer which is permanently attached at one end to a supporting surface. The graft polymer may be comprised of a single monomeric repeat unit or of two or more different monomer units distributed along the length of the polymer chain in either an ordered or random manner.
Backbone: The main monomeric repeat unit is considered the graft polymer backbone along with the unsaturated portions of the other comonomers present that react to form the graft polymer chain.
Active chemical moieties: Chemical functional groups that are attached to the graft polymer backbone. These functional groups may, if desired, be attached as side chains to the backbone of the graft polymer. Also if desired, these sites may be reacted with other molecules to form new graft side-chain moieties with chemical reactivity that is either the same as or different from that of the original active site. Active sites are the points at which chemical or biochemical probes or targets are attached to the graft polymer.
Side chain spacer arm: The chain of atoms separating the active chemical moiety and the graft polymer backbone.
Structural modifier: A polymer side chain which functions to fine tune assay performance. Examples include inert or charged groups which control the hydrophilicity, pH, charge and structure of the assay matrix.
Probe: A chemical or biochemical species that is attached to an active site on a graft polymer. Probes have inherent specificity for binding to chemical or biochemical target molecules or molecular fragments. The specificity of the probe for a particular target may, if desired, be a function of target composition, molecular structure and/or conformation or chemical or biochemical function.
Target: A chemical or biochemical species that is the subject of an assay experiment. Examples of targets are viruses, bacteria, DNA, RNA, proteins, lipids, toxins, or other chemical or bioactive agents for which information on their presence or concentration in a sample is of interest. A target may bind to the probes or directly to the active chemical moiety. For example, a therapeutic drug may bind directly to the active chemical moiety
Sensitivity: The minimum signal that is detectable from background.
Dynamic range: The range of signal that spans from the maximum signal that is discernible from saturation to the minimum signal that is discernible over background.
Initiator: A compound that generates reactive radical sites on the surface of the substrate and is thereby capable of initiating a graft polymerization reaction on the substrate.
Primer: A coating that is applied to a surface that will allow radical initiator molecules to be immobilized on said surface. The immobilized radical initiator molecules can be activated and used to initiate a graft polymerization reaction on said primer-coated surface. The resulting graft polymer should be permanently attached to the primer layer and thus, by extension, attached to the surface. The primer layer must have sufficient mechanical integrity and chemical inertness to remain intact throughout the graft polymerization reaction.
Primer solution: A solution of primer dissolved in a common solvent or solvent mixture. In one embodiment of the invention, this solution is applied to the surface upon which the graft polymer is to be grown. A second step, comprising applying a solution of initiator in a solvent or solvent mixture is subsequently used to deliver initiator to said surface.
Initiator/Primer: A mixture of primer and initiator. The mixture may exist in the presence or absence of a mutual solvent or solvent mixture. In one embodiment of the present invention it is the vehicle for delivering initiator to the primer-coated surface to make it amenable to graft polymerization.
Silane Monomer: A reactive monomer with a silane group attached to it that can bind to a glass surface.
Silane Initiator: A radical initiator with a silane group attached to it that can bind to a glass surface.

Generally, the invention relates to a graft polymer three-dimensional non-crosslinked matrix coating with a controlled graft polymer chain surface density and controlled chain length and a method for achieving said coating to which chemical or biochemical probe molecules may be attached, said probe molecules having inherent specificity for binding to target chemical or biochemical species. The following discussion presents details of features of the invention.

a) Delivery of Initiator to the Surface of an Article

U.S. Pat. No. 6,358,557 discloses the concept of attaching a graft polymer to the surface of an article by first dipping the article into a solution containing a solution of initiator and subsequently performing a graft polymerization reaction upon the initiator-treated surface. There are some surfaces such as glass, metal, and some polymers that are incapable of swelling or accepting radical transfer from an initiator molecule, thus making surface graft polymerization impossible. The present invention teaches three methods by which such a radical-insensitive surface may be rendered suitable for graft polymerization.

One method is to apply to the unreactive surface a primer layer which may subsequently be exposed to a medium containing initiator molecules. When exposed to the initiator containing medium, the primer layer should swell sufficiently to allow initiator molecules to penetrate the coating, yet retain sufficient mechanical integrity that it does not detach from the surface or dissolve in the initiator containing medium. The second method is to apply to the unreactive surface, an initiator/primer layer. This may be accomplished by co-dissolving the initiator and primer into a medium, typically an organic solvent or mixture of solvents and then applying said coating to the surface to be graft polymerized. In this way, the initiator may be applied to the surface without the need for a subsequent coating/swelling step. The third method is to attach an initiator to a glass surface using a silane group attached to the initiator.

Examples of suitable initiators can be found in U.S. Pat. No. 6,358,557. Peroxides, azo initiators, redox initiators, photoinitiators and photosensitizers can be used in this process. Thermal initiators, including peroxide and azo initiators, and redox initiators can be used to perform graft polymerization on the inner lumen surface if the devices are hollow as well as on the outer surface of a substrate. Using these initiators, both the free radicals and monomers in the liquid medium can access the lumen and perform graft polymerization under appropriate initiation conditions. Thermal initiators may give relatively constant initiation rates during the process, while the initiation rate for redox initiators declines quickly because of the rapid consumption of initiator components. The initiation by radiation, with and without photolytic initiators, is limited in lumens, since the radiation intensity is restricted or reduced while penetrating through the substrate wall.

Peroxide initiators include but are not limited to: peroxyesters, such as 1,1-dimethyl-3-hydroxybutyl peroxyneodecanoate, α-cumyl peroxyneodecanoate, α-cumyl peroxyneoheptanoate, t-amyl peroxyneodecanoate, t-butyl peroxyneodecanoate, t-amyl peroxypivalate, t-butyl peroxypivalate, 2,5-dimethyl 2,5-di(2-ethylhexanoylperoxy)hexane, t-butylperoxy-2 ethylhexanoate, t-butylperoxyacetate, t-amylperoxyacetate, t-butylperbenzoate, t-amylperbenzoate, t-butyl 1-(2-ethylhexyl)monoperoxycarbonate, and others; peroxyketals, such as, 1,1-di(t-butylperoxy)-3,3,5-trimethyl-cyclohexane, 1,1-di(t-butylperoxy)-cyclohexane, 1,1-di(t-amylperoxy)-cyclohexane, ethyl-3,3-di(t-butylperoxy)-butyrate, ethyl-3,3-di(t-amylperoxy)-butylperoxy)-butylrate, and others; peroxydicarbonates, such as di(n-propyl)perosydicarbonate, di(sec-butyl)perosydicarbonate, di(2-ethylhexyl)perosydicarbonate, and others; ketone peroxides, such as, 2,4-pentanedione peroxide, and others; hydroperoxides, such as cumene hydroperoxide, butyl hydroperoxide, amyl hydroperoxide, and others; dialkyl peroxides, such as, dicumyl peroxide, dibutylperoxide, diamylperoxide, and others; diacyl peroxide, such as, decanoyl peroxide, lauroyl peroxide, benzoyl peroxide, and others; inorganic peroxides, such as hydrogen peroxide, potassium persulfate, and others. Azo initiators include, for example, azobisisobutyronitrile, azobiscumene, azo-bisiso-1,1,1-tricyclopropylmethane, 4-nitrophenyl-azo-triphenylmethane, phenyl-azo-triphenylmethane, and others. Redox initiators include, but are not limited to peroxide-amine systems, peroxide-metal ion systems, boronalkyl-oxygen systems, and others. Photoinitiators/photosensitizers include, but are not limited to, organic peroxide and azo initiators, benzophenone, benzophenone derivatives, camphorquinone-N,N dimethyl-amino-ethyl-methacrylate, and others.

Examples of suitable silane initiators are 1,1-diphenylethylene-chlorosilane, 2-(4-chlorosulfonylphenyl) ethyl trimethoxysilane,azobis isobutyronitrile 2-(acryloxethoxy)trimethylsilane and the like could be used.

b) Graft Polymerization

Any method of graft polymerization know to those skilled in the art could be used to practice the invention. The general method used for preparing the experimental examples is a reversed phase graft polymerization process described in U.S. Pat. No. 6,358,557. Optionally, the reaction medium in which the graft polymerization is performed can have as an additional component a hydrophilic, water soluble polymer such as, for example, poly(vinylpyrrolidone). Preferably the concentration of the water-soluble, hydrophilic polymer will be between about 0 and about 10 percent of the solids contained in the reactive medium.

c) Composition of the Graft Polymer Layer

The graft polymer layer is typically a linear or branched polymer comprising one or more distinct monomer units. A wide range of monomers may be used in the graft polymerization reaction. Free radical polymerizable monomers or oligomers can be used in this process based on the their hydrophilicity and the required surface modification. Generally, vinyl monomers, particularly, acrylic monomers, are useful because the high solubility of these monomers leads to easy operation in a wide range of monomer concentrations. Useful hydrophilic monomers include but are not limited to: hydroxyl substituted ester acrylate and ester methacrylate, such as 2-hydroxyethylacrylate, 2- and 3-hydroxypropylacrylate, 2,3-dihydroxypropylacrylate, polyethoxyethyl-, and polyethoxypropylacrylates; acrylamide, methacrylamide and derivatives, such as, N-methylacrylamide, N,N-dimethylacrylamide, N,N-dimethylmethacrylamide, N,N-dimethyl- and N,N-diethyl-aminoethyl, 2-acrylamido-2-methyl-1-propanesulfonic acid, N-[3-dimethylamino)propyl]acrylamide, 2-(N,N-diethylamino)ethyl methacrylamide, and others; poly(ethylene glycol) acrylates, poly(ethylene glycol) methacrylates, poly(ethylene glycol) diacrylates, poly(ethylene glycol) dimethacrylates; poly(propylene glycol) acrylates, poly(propylene glycol) methacrylates, poly(propylene glycol) diacrylates, poly(propylene glycol) dimethacrylates; acrylic acid, methacrylic acid and the substituted; 2- and 4-vinylpyridine; 4- and 2-methyl-5-vinylpyridine; N-methyl-4-vinylpiperidine; 2-methyl-1-vinylimidazole,; dimethylaminoethyl vinyl ether; N-vinylpyrrolidone; itaconic, crotonic, fumaric and maleic acids, and others.

Hydrophobic monomers include but are not limited to ester acrylates and ester methacrylates such as methyl, ethyl, propyl, butyl, phenyl, benzyl, cyclohexyl, ethoxyethyl, methoxyethyl, ethoxypropyl, hexafluoroisopropyl or n-octyl-acrylates and -methacrylates; acrylamides and methacrylamides; dimethyl fumarate, dimethyl maleate, diethyl fumarate, methyl vinyl ether, ethoxyethyl vinyl ether, vinyl acetate, vinyl propionate, vinyl benzoate, acrylonitrile, styrene, alpha-methylstyrene, 1-hexene, vinyl chloride, vinyl methyl ketone, vinyl stearate, 2-hexene and 2-ethylhexyl methacrylate.

Preferably, at least one of the monomers that comprises the graft polymer contains one or more functional groups that do not react during the polymerization process and can therefore retain their functionality while being attached to the graft polymer chain. More preferably, these functional groups will have the ability to react with and chemically bond to other molecules or molecular fragments. General examples of the types of reactivity that may be incorporated into the graft polymer chain are carboxylic acids, alkyl or aromatic amines, aldehydes, thiols, maleimides, alkyl or aryl iodides, functional groups that are susceptible to nucleophilic attack, and functional groups that are capable of forming ionic interactions. There are many examples known to those skilled in the art of monomers that have functional groups that can become side-chains in a graft polymer. Representative examples of such monomers are acrolein, 2-bromoacrylate, dicyclopentenyl acrylate, 2-(N,N,-diethylamino)ethyl methacrylate, 2-(ethylthio)ethyl methacrylate, n-hexylmethacrylate. Numerous suitable monomers are available commercially or can be synthesized according to methods known in the art.

Five representative examples of co-monomers with suitable active sites are 3-aminopropylacryalmide and 2-aminoethyl methacrylate (which have a side-chain amine group) and acrylic acid, beta-carboxyethyl acrylate, beta-acryloyl oxyethyl hydrogen succinate (which have a side-chain carboxylic acid group). Co-monomers like beta-acryloyl oxyethyl hydrogen succinate having side-chain carboxylic acid moieties are especially preferred. Optionally, the side-chain carboxylic acid functionality may be modified to create a new active site with enhanced reactivity to a desired probe. One preferred method for activating side-chain carboxylic acid functional groups is the stepwise reaction of the free carboxylic acid side-chain functionality with a dehydrating agent and N-hydroxysuccinimide (NHS) to form an activated succinimidyl ester side-chain species. There are a variety of reagent combinations known to one skilled in the art to achieve such a coupling reaction. One preferred combination is the reaction of the side-chain carboxylic acid with 1-ethyl-3-(3-dimethylaminopropyl) carbodiimide hydrochloride and N-hydroxysuccinimide. The resulting graft polymer containing activated succinimidyl side-chain species may be washed, dried and stored under dry conditions for later use. This NHS-carboxylic acid ester may subsequently be reacted with a probe containing an amine group and therefore, used to attach a probe molecule to the graft polymer side-chain.

The fraction of monomeric repeat units in a graft polymer that have the ability to chemically bond to other probe or target molecules may be chosen to maximize the number density of probes or targets within the three dimensional matrix. For example, if the graft polymer matrix is to be used to bind relatively large probes or targets, then the fraction of monomers with active site functionalities may desirably be less than the fraction that would be used in a graft polymer matrix that is to be used to bind relatively small probes or targets. Preferably the mole fraction of monomers with such active sites is between about 0.01 and 50 mole percent. For other applications where the target and probe species are relatively small, the preferable mole fraction of active site functionalities may be between about 5 and 100 percent. It is known in the art that monomers react at different rates. Thus, the starting mole percents of reactant monomers don't necessarily represent the final graft polymer composition. One skilled in the art can compensate for these different monomer reaction rates, for example, by changing the starting mole percents of the reactive monomers or changing the reaction conditions, such as time, temperature, salt concentration, initiator, etc. to arrive at or near a desired final graft polymer matrix structure.

Optionally, one of the co-monomers has attached to it an oligomeric chain that can function as a structural modifier when the monomer is incorporated into a graft polymer. Preferably the structural modifier provides a mechanism for incorporating transient entanglements into the three-dimensional structure of the graft-polymer matrix. Transient entanglements offer a means of controlling the morphology and robustness of the three dimensional coating without incorporating permanent crosslinks into the matrix that might be expected to adversely affect the diffusion of target species through the matrix.

The structural modifier may also optionally be chosen to provide an optimal level of hydrophilicity to the three-dimensional matrix. Control of the coating hydrophilicity is important for creating an appropriate milieu for probe or target molecules to exist in their native state without denaturing. There are many examples of monomers that have oligomeric or polymeric species that can function as a structural modifier when incorporated into a graft polymer. Representative examples of such monomer species are a series of poly(ethylene glycol) acrylates and methacrylates produced commercially by Laporte Performance Chemicals, UK and sold under the Bisomer name. These monomers have as side-chains oligomeric ethylene oxide repeat units with average molecular weights of between about 42 and 2000 g/mole. The combination of side-chain composition and molecular weight and the mol fraction of monomeric repeat units containing oligomeric side-chain structural modifiers may be chosen by one skilled in the art to optimize diffusivity, flexibility, hydrophilicity and robustness of the coating.

Optionally, the structural modifier may also be chosen so that it is attached permanently to two or more graft polymer chains. This may be done, for example, by incorporating a difunctional co-monomer into the graft polymerization reaction medium. Representative difunctional monomers suitable for this purpose are series of poly(ethylene glycol) diacrylates and dimethacrylates produced commercially by Laporte Performance Chemicals, UK and sold under the Bisomer name. These monomers have as side-chains oligomeric alkylene oxide repeat units with average molecular weights of between about 42 and 1200 g/mole. Structural modifiers incorporated in this manner might be expected increase the robustness the resulting graft polymer coating, with said robustness improving the utility of the coating. Because it does introduce some level of inter-graft connectivity, analogous to a crosslink, but less rigid than typical or highly crosslinked polymer systems, coatings prepared in this way may be less suitable for some applications than those coatings prepared without the bifunctional structural modifier.

Optionally, one or more of the co-monomers may be chosen to alter the manner in which the coating interacts with liquids and vapors. Preferably it also provides a moist, hydrogel medium with solution properties that prevent species like some proteins from denaturing during storage. For example, co-monomers with hydrophobic side chains could be incorporated to increase the hydrophobic nature of the coating. Alternatively, co-monomers having relatively more hydrophilic groups could be incorporated to increase the hydrophilic nature of the coating. Alternatively, co-monomers with ionic side chains may be incorporated as well.

Optionally the choice or mixing of backbone monomers can be used to tailor the hydrophilic nature of the graft polymer matrix.

Optionally, one or more of the co-monomers may be chosen to have diagnostic image enhancement qualities.

Optionally, one or more of the co-monomers may be chosen to have biological activity. Representative examples of such biological activities include anti-infective properties, anti-thrombogenic properties, cytotoxicity, therapeutic properties, biochemical inhibition, agonist properties, antagonist properties, or prodrug properties.

The concentration of these co-monomers may be chosen to optimize the distribution of reactive sites throughout the graft-polymer matrix.

d) Attachment of Probe Groups to Graft Co-Polymer

Preferably, the active sites on the graft polymer chain may be further coupled to probe species having inherent specificity for binding to chemical or biochemical target molecules or molecular fragments. Preferred probe or target species include viruses, bacteria, fungi, parasites, DNA, RNA, monoclonal antibodies, single chain antibodies and/or peptides, or antigens and/or protein-markers, lipids, toxins, or other bioactive agents for which information on their presence or concentration in a sample is of interest.

Optionally, the active sites on the graft polymer chain may be further coupled to molecules or molecular fragments that will impart specific functional utility to an article coated with the coating. A wide variety of functional utilities can be envisioned by the invention. One such functional utility would be the ability for the coated article to be more easily detected by techniques like fluorescence spectroscopy, magnetic resonance, computer tomography, x-ray radiation, ultrasound radiation microwave radiation or the like relative to an uncoated article. Alternatively, the functional utility could impart anti-infective or anti-cytotoxic properties to a coated article that are superior to those of an uncoated article.

Optionally, a mixture of probes, each having its own specificity to a target, may be co-printed onto a graft-polymer surface to allow for multiple target analysis at the same geographic surface location. This may be accomplished, for example, by mixing respective amino containing probes together and printing said mixture onto a NHS-activated graft polymer coated surface. Alternatively, different functional groups may be incorporated onto the polymer chain, for example, amino or sulfhydryl groups, followed by the printing of probes containing specific reactivities to these groups, for example, NHS or iodoacetyl groups, respectively. Different fluorescent labels can be tagged to each respective probe to allow for the detection and quantification of multiple target species in the same printed area.

e) Control of Graft Polymer Surface Density and Chain Length.

The density of graft chains upon a surface is dependent upon the concentration and distribution of initiator on the surface as well as by controlling the ratio of reactive and nonreactive polymers. In the present invention, variation of the initiator concentration may be accomplished by modifying the concentration of the initiator in the medium into which the primed surface is immersed. Alternatively initiator concentration may be varied by modifying the relative concentrations of initiator and primer in a medium containing both. Graft polymerization of surfaces primed with a lower concentration of initiator in the initiator solution or initiator/primer solution result in surfaces with distinct differences from those primed with solutions containing higher concentrations of initiator. First, we found that coating opacity was directly related to the concentration of initiator used and that opacity increases with corresponding increases in the surface density of initiator thus resulting in an increase in the surface density of the graft polymer chains. By choosing an optimal concentration of initiator in the initiator or initiator/primer solution, we produced graft polymer coated surfaces that were substantially transparent. Secondly, when we compared the two means of introducing initiator to the surface, dipping primer-coated slides in initiator solution and dipping uncoated slides in a solution of primer and initiator, we saw, surprisingly different results in terms of distribution of initiator on the surface and the robustness of the primer to subsequent processing steps. The resulting graft polymer layer was more uniform on slides that were coated with the initiator/primer mixture. Since the initiator and primer are applied in one step, this method is also likely to be more easily scaled than a two-step process. We found, however, that the adhesion to glass substrates of the initiator/primer layer was degraded somewhat relative to primer containing no initiator. Thus, a combination process comprising first coating the substrate with pristine primer and subsequent coating with a second layer of an initiator/primer may be preferred.

The density of graft polymer chains upon a surface is also dependent upon the concentration and distribution of initiator reactive groups on the surface. Thus by mixing polymers with and without reactive groups and exposing them to, or mixing initiator in with, the polymers, the graft polymer surface density can be controlled. The spacing of graft polymer chains can also be achieved by mixing together silanes with reactive monomers attached and silanes with unreactive groups attached. The same control of chain density can be achieved by mixing silanes with initiators attached and silanes with unreactive groups attached. Control of graft polymer chain density could also achieved by exposing an unreactive surface to radiation that would produce initiator reactive sites on the surface. By changing the type, intensity and duration of the radiation the number and distribution of the initiator reactive sites could be controlled.

The thickness, or chain length, of the three-dimensional graft polymer layer may be controlled by modifying the length of time that the surface is exposed to the graft polymerization reaction conditions. As reaction times increase, graft polymer chain lengths and total graft polymer coating thicknesses will increase proportionally.

f) Attachment of Monomers to a Surface

Reactive monomers can also be attached to a surface. For example methacrylate and acrylate functional siloxanes can be used to attach reactive monomers to glass surfaces. Examples of silanes with attached reactive monomers are (3-acryloxypropyl)trimethoxy-silane, (3-acryloxypropyl)tris(trimethyl-siloxy)silane, (3-acryloxypropyl)trichloro-silane, methacryloxypropyltrimethoxy-silane, methacryloxypropyltrichloro-silane, methacryloxypropyltris(methoxy-ethoxy)silane, methacryloxypropyltriethoxy-silane, and the like.

### EXAMPLES

### Example 1

### Preparation of a Graft Polymer Layer Containing Acrylamide, Aminopropylacrylamide, and PEG-Methacrylate with Initiator Incorporated into the Primer

Application of Primer

A solution of primer containing a radical initiator was prepared by mixing together a styrene-butadiene based adhesive (Eclectic Products, Springfield Oreg.), 4.8 g, tetrahydrofuran, 21.5 g, toluene, 21.5 g, and lauryl peroxide, 0.24 g. The concentration of lauryl peroxide was 10 percent by weight of solids. Five standard sized (25 mm×75 mm×1.1 mm) glass microscope slides that had been previously cleaned by washing with tetrahydrofuran were dipped approximately 5 cm into and removed from the initiator/primer solution and dried at room temperature overnight.

Application of Graft Polymer

A concentrated aqueous solution of sodium chloride was prepared by dissolving sodium chloride, 290 g, and poly(vinylpyrrolidone) (K90 grade, BASF), 4.0 g, in deionized water, 1710 g. A reactive monomer solution was prepared by next dissolving acrylamide, 5.84 g, S-20W poly(ethylene glycol) methacrylate, 3.84 g, 3-aminopropylacrylamide hydrochloride, 2.87 g in 480 g of the salt solution.

Five glass microscope slides were stacked together, placing a 2 cm piece of microscope slide between each slide to keep them separated, and then placed in a reactor vessel along with 123 g of the monomer solution. The reaction tube was degassed by twice repeated evacuation and flushing with nitrogen gas. The reactor system was equilibrated to atmospheric pressure of nitrogen and immersed in a preheated water bath (89° C.) for 30 minutes. The reactor was removed from the water bath and cooled. The coated slides were rinsed with copious amounts of water and allowed to dry at ambient conditions.

In this manner, slides were prepared having a graft polymer comprising polyethylene glycol spacer arms and alkylamine active groups attached to the graft polymer backbone.

### Example 2

### Preparation of a Graft Polymer Layer Containing Acrylamide, Aminopropylmethacrylamide, and PEG-Methacrylate with a Two-Step Primer Coating and Initiator Application

Application of Primer

A solution of primer was prepared by mixing together a styrene-butadiene based adhesive (Eclectic Products, Springfield Oreg.), 4.9 g, tetrahydrofuran, 22 g, and toluene, 22 g. This solution was filtered through a nylon filter screen with a pore size of 75 μm. Five standard sized (25 mm×75 mm×1.1 mm) glass microscope slides that had been previously cleaned by washing with tetrahydroftiran were dipped approximately 5 cm into and removed from the solution of primer and dried at 60° C. under dynamic vacuum. A solution of radical initiator was prepared by dissolving lauryl peroxide, 0.20 g, in acetone, 49 g. Each of the primer coated slides was subsequently dipped approximately 5 cm into and removed from the solution of initiator and allowed to dry at room temperature overnight.

Application of Graft Polymer

A concentrated aqueous solution of sodium chloride was prepared by dissolving sodium chloride, 150 g, in deionized water, 850 g. A reactive monomer solution was prepared by next dissolving acrylamide, 4.46 g, S-20W polyethylene glycol methacrylate, 3.84 g, 3-aminopropylmethacrylamide hydrochloride, 0.47 g in 192 g of the salt solution.

Five glass microscope slides were assembled together and placed in a reactor vessel along with the monomer solution as described in Example 1. The reaction tube was degassed by twice repeated evacuation and flushing with nitrogen gas. The reactor system was equilibrated to atmospheric pressure of nitrogen and immersed in a preheated water bath (89° C.) for 45 minutes. The reactor was removed from the water bath and cooled. The coated slides were rinsed with copious amounts of water and allowed to dry at ambient conditions.

In this manner, slides were prepared having a graft polymer comprising poly(ethylene glycol) spacer arms and alkylamine active groups attached to the graft polymer backbone.

### Example 3

### Reaction of an N-Hydroxysuccinimide Probe with an Amine-Containing Graft Polymer Modified Surface (Colorimetric Analysis)

Serial dilutions of N-hydroxysuccinimide-modified biotin (NHS-biotin, Pierce) were prepared by dissolving the appropriate amount of NHS-biotin in PBS. In all, ten solutions were prepared, one each having a concentration of NHS-biotin of 500, 250, 125, 63, 31, 16, 8, 4, 2, and 1 μg/ml.

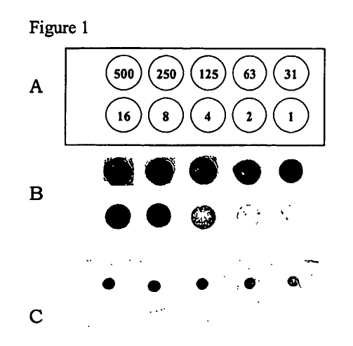

A slide from Example 1, containing side-chain amine active groups was spotted in distinct areas with 1 μl of each NHS-biotin solution following the pattern described in FIG. 1a. The slide was incubated for 30 minutes in a humidity chamber at room temperature and washed in PBS solution for 5 minutes. The slide was immersed in a solution of streptavidin-alkaline phosphatase (25 μg/ml, Pierce) for 30 minutes at room temperature. The slide was developed with NBT/BCIP precipitating substrate (Pierce) for 15 minutes followed by washing with deionized water. The slide was imaged on an Olympus AX70 microscope with a RT SPOT digital camera. Individual spots were imaged at 4× and imported into Adobe Photoshop® for the creation of a composite image, shown in FIG. 1b.

FIG. 1b shows an increase in color density that corresponds to differing concentrations of NES-biotin available for binding to streptavidin on the surface from 1 to 31 μg/ml. The range of spot concentrations from 63 to 500 μg/ml that show no change in spot intensity because the amount of NHS-biotin in each of those spots exceeded the number of reactive amine sites available in the printed area.

### Example 4

### Reaction of a N-Hydroxysuccinimide-Probe with Amine Containing Graft Polymer Surface (Fluorescence Analysis)

Serial dilutions of N-hydroxysuccinimide-modified biotin (NHS-biotin, Pierce) were prepared as described in Example 3. A slide from Example 1, containing side-chain amine active groups, was spotted in distinct areas with 1 μl of each NHS-biotin solution following the pattern described in FIG. 1a. The slide was incubated for 30 minutes in a humidity chamber at room temperature and washed in PBS solution for 5 minutes. The slide was immersed in a solution of streptavidin-Cy3 (25 μg/ml, Pierce) for 30 minutes at room temperature and then dried. The slide was imaged by scanning in a Virtek ChipReader at a 20 μm resolution with a laser and PMT setting defaulted to maximum levels.

FIG. 1C shows an increase in color density (fluorescence) that corresponds to differing amounts of NHS-biotin available for binding to streptavidin on the surface from 1 to 31 μg/ml. The range of spot concentrations from 63 to 500 μg/ml that show no change in spot intensity because the amount of NHS-biotin in each of those spots exceeded the number of reactive amine sites available in the printed area.

### Example 5

### Preparation of the Graft Polymer Layer Containing Acrylamide, Acrylic Acid, and PEG-Methacrylate

Application of Primer

A solution of primer containing a radical initiator was prepared by mixing together a styrene-butadiene based adhesive (45% solid, Eclectic Products, Springfield Oreg.), 10.0 g, tetrahydrofuran, 44.8 g, toluene, 44.8 g, and lauryl peroxide, 0.45 g. The concentration of lauryl peroxide was 10 percent by weight of solids. Five standard sized (25 mm×75 mm×1.1 mm) microscope slides that had been previously cleaned by washing with tetrahydrofuran were dipped approximately 5 cm into and removed from the initiator/primer solution and dried at room temperature overnight.

Application of Graft Polymer

A concentrated aqueous solution of sodium chloride was prepared by dissolving sodium chloride, 55 g, in deionized water, 425 g. A reactive monomer solution was prepared by next dissolving acrylamide, 4.80 g, S-20W polyethylene glycol methacrylate, 2.64 g, acrylic acid, 0.96 g in 240 g of the salt solution.

Five microscope slides were stacked together, placing a 2 cm piece of microscope slide between each slide to keep them separated. A portion of the monomer and salt solution prepared above, 124 g, and the slides were placed in a glass tube (6 cm×40 cm). The reaction tube was degassed by twice repeated evacuation and flushing with nitrogen gas. The reactor system was equilibrated to atmospheric pressure of nitrogen and immersed in a preheated water bath (89° C.) for 45 minutes. The reactor was removed from the water bath and cooled. The coated slides were rinsed with copious amounts of water and allowed to dry at ambient conditions.

In this manner, slides were prepared having a graft polymer comprising polyethylene glycol spacer arms and acrylic acid carboxylic acid active groups attached to the graft polymer backbone.

### Example 6

### General Method for Activation of Graft Polymer Carboxylic Acid Active Sites with N-Hydroxysuccinimide (NHS)

A solution was prepared by mixing 0.1 M MES buffer (2-[N-morpholino]ethanesulfonic acid, Sigma), 100 ml and 0.5 N aqueous sodium chloride, 100 ml. The pH of this solution was adjusted to pH 6 by addition of and aqueous solution of 0.8 M sodium hydroxide. pH measurement was performed using an Accumet pH Meter 50 (Fisher Scientific). To a 50 ml aliquot of this solution was added 1-ethyl-3-(3-dimethylaminopropyl) carbodiimide hydrochloride (EDC, Pierce), 0.020 g, and N-hydroxysuccinimide (Aldrich), 0.030 g. Primer-coated slides from Example 5 were immersed in the EDC/NHS solution and stirred for 15 minutes. Slides were removed from this solution, immersed for 1 minute in deionized water, and dried under vacuum. NHS-activated slides were stored at room temperature under dry conditions until further use.

### Example 7

### Reaction of an Amine-Containing Probe with a NHS-Modified Graft Polymer Modified Surface

Serial dilutions of biocytih and biotin were prepared using biocytin (Pierce) and biotin (Pierce) dissolved in the appropriate amount of phosphate buffered saline solution (PBS, 0.1 M sodium phosphate and 0.15 M sodium chloride , pH 7.2). In all, ten solutions were prepared. Five solutions had biotin at concentrations of 2.0, 1.0, 0.5, 0.25, 0.125, and 0.0625 mM. Five solutions had biocytin at concentrations of 2.0, 1.0, 0.5, 0.25, 0.125, and 0.0625 mM.

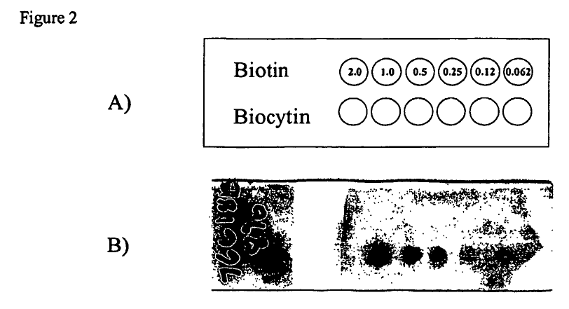

A slide from Example 5 and activated with N-hydroxysuccinimide following the procedure of Example 6 was spotted in distinct areas with 2 μl of each of the solutions of biocytin and biotin prepared above following the pattern described in FIG. 2a. The slide was incubated for 1 hour in a humidity chamber at room temperature. After washing in PBS, the slide was blocked for 0.5 hour in Tris/gly buffer (0.1 M Tris/0.05 M glycine/0.15 M sodium chloride at pH 7.5) to cleave any remaining unreacted N-hydroxysuccinimidyl ester. The slide was washed with Tris/gly buffer (1 change, 25 ml). The slide was immersed in a solution of streptavidin-alkaline phosphatase (Pierce, diluted to 0.025 mg/ml in Tris/gly buffer) for 30 minutes at room temperature. The slide was then washed 4 times, 5 minutes each, with PBS solution. The slide was developed with NBT/BCIP precipitating substrate (Pierce) for 15 minutes followed by washing with deionized water. The slide was optically scanned on a flatbed scanner, an image of which is shown in FIG. 2b.

FIG. 2 shows an increase in color density that corresponds to an increase in printed biocytin concentration across the graft polymer surface. The series of biotin spots show no color development, consistent with the fact that biotin does not have a primary amine available for binding with N-hydroxysuccinimide activated carboxylic acid groups attached to the graft polymer.

### Example 8

### Determination of N-Hydroxysuccinimide Concentration on a Surface

A solution of containing 0.01 mg/ml N-hydroxysuccinimide in PBS at pH 7.2 was prepared and the ultraviolet absorption properties measured using a Shimadzu spectrophotometer. The molar extinction coefficient was determined to be approximately 8500 at 260 nm. The surface area of coating on a slide from Example 5 and activated with N-hydroxysuccinimide following the procedure of Example 6 was measured. The slide was placed into 18 ml of 0.05 M NaOH for 4 hours at room temperature. The slides were removed and 2 ml 10×PBS was added to the solution. The pH of the solution was adjusted to 7.3 with 2 drops of concentrated aqueous 1M hydrochloric acid. The solution was scanned with a spectrophotometer. The absorbance value at 260 nm was measured and divided by the molar extinction coefficient determined from the standard to determine the total amount of NHS in solution. The concentration of NHS per unit area was then determined to be 0.25 μg/cm² by dividing the total amount of NHS by the area of coated surface.

### Example 9

### Preparation of a Graft Polymer Layer Containing Acrylamide, and PEG-Methacrylate Using a Silicone Based Precoat Layer

Application of Primer

A solution of primer was prepared by mixing together a silicone based adhesive (MasterSil 415, Master Bond Inc., Hackensack N.J.), 4.68 g and tetrahydrofuran, 42.34 g. Five standard sized (25 mm×75 mm×1.1 mm) microscope slides that had been previously cleaned by washing with tetrahydrofuran were dipped approximately 5 cm into and removed from the solution of primer and dried at room temperature overnight. A solution of radical initiator was prepared by dissolving lauryl peroxide, 3.24 g, and acetone, 80.01 g. Each of the primer coated slides was subsequently dipped approximately 5 cm into and removed from the solution of initiator and allowed to dry at room temperature for one hour.

Application of Graft Polymer

A concentrated aqueous solution of sodium chloride was prepared by dissolving sodium chloride, 290 g, and poly(vinylpyrrolidone) (K90 grade, BASF), 4.0 g, in deionized water, 1710 g. A reactive monomer solution was prepared by next dissolving acrylamide, 3.80 g, S-20W poly(ethylene glycol) methacrylate, 2.43 g, in 240 g of the salt solution.

The five microscope slides were assembled together with glass slide spacers between each one to keep them separated. This assembly was placed in a reactor vessel along with 123 g of the monomer solution described above. The reaction tube was degassed by twice repeated evacuation and flushing with nitrogen gas. The reactor system was equilibrated to atmospheric pressure of nitrogen and immersed in a preheated water bath (89° C.) for 20 minutes. The reactor was removed from the water bath and cooled. The coated slides were rinsed with copious amounts of water and allowed to dry at ambient conditions.

In this manner, slides were prepared using a silicone primer having a graft polymer comprising polyethylene glycol spacer arms attached to the graft polymer backbone of acrylamide units. The graft polymer layer was clearly visible after the water rinsing and room temperature drying.

### Example 10

### Preparation of a Graft Polymer Layer Containing Acrylamide, and PEG-Methacrylate Using an Acrylic Copolymer Based Precoat Layer

Application of Primer

A solution of primer was prepared by mixing together an acrylic polymer Paraloid AT-746 (Rohm and Haas, Philadelphia Pa.), 0.16 g, a melamine—formaldehyde resin CYMEL 248-8 (Cytec, West Paterson N.J.), 0.05 g, a acrylic copolymer Acryloid B-48N (Rohm and Haas, Philadelphia Pa.), 0.32 g, acetic acid 0.01 g, toluene 0.95 g, and methyl ethyl ketone 10.55 g. Five standard sized (25 mm×75 mm×1.1 mm) microscope slides that had been previously cleaned by washing with tetrahydrofuran were dipped approximately 5 cm into and removed from the solution of primer and dried at 110 C for 1 hour. A solution of radical initiator was prepared by dissolving lauryl peroxide, 3.24 g, and acetone, 80.01 g. Each of the primer coated slides was subsequently dipped approximately 5 cm into and removed from the solution of initiator and allowed to dry at room temperature for one hour.

Application of Graft Polymer

A concentrated aqueous solution of sodium chloride was prepared by dissolving sodium chloride, 290 g, and poly(vinylpyrrolidone) (K90 grade, BASF), 4.0 g, in deionized water, 1710 g. A reactive monomer solution was prepared by next dissolving acrylamide, 1.91 g, S-20W poly(ethylene glycol) methacrylate, 1.28 g, in 120.17 g of the salt solution.

The five microscope slides were assembled together with 2 cm glass slide spacers between each one to keep them separated. This assembly was placed in a reactor vessel along with 123 g of the monomer solution described above. The reaction tube was degassed by twice repeated evacuation and flushing with nitrogen gas. The reactor system was equilibrated to atmospheric pressure of nitrogen and immersed in a preheated water bath (89° C.) for 30 minutes. The reactor was removed from the water bath and cooled. The coated slides were rinsed with copious amounts of water and allowed to dry at ambient conditions.

In this manner, slides were prepared using an acrylic copolymer primer having a graft polymer comprising poly(ethylene glycol) spacer arms attached to the graft polymer backbone of acrylamide units. The graft polymer layer was clearly visible after the water rinsing and room temperature drying.

### Example 11

### Determination of Coating Thickness and Mass

Solution preparation. A solution of initiator (3.85 percent by weight) was prepared by dissolving lauryl peroxide, 9.62 g, in tetrahydrofuran, 120 g and acetone, 120 g. A concentrated aqueous solution of sodium chloride was prepared by dissolving sodium chloride, 290 g, and poly(vinylpyrrolidone), 4 g, in deionized water, 1710 g. A reactive monomer solution was prepared by dissolving acrylamide, 6.08 g, and S-20W polyethene glycol methacrylate, 3.80 g, in 380 g of the salt solution.

Substrate coating. Medical grade colorless silicone tubing (Helix Medical, 3.18 mm outer diameter×1.58 mm inner diameter) was cut into segments approximately 21 cm long. For each run, fifteen segments were taken together and their mass measured. The segments were immersed in the initiator solution, for 60 s, removed and then dried in air for one hour. The samples were subsequently placed in the reactor described in Example 1 of the present application, a glass tube (6 cm×40 cm) along with the monomer/salt solution. The reaction mixture was degassed by twice repeated evacuation and flushing with nitrogen gas. The reactor system was equilibrated to atmospheric pressure of nitrogen and immersed in a preheated water bath (89° C.) for 30, 45, or 60 minutes. The reactor was removed from the water bath and cooled. The coated tubes were rinsed with copious amounts of water and allowed to dry at ambient conditions and then re-weighed. The initial and final values for the total tubing mass before and after graft polymerization, as well as percentage weight gain, are shown as a function of reaction time in Table 1. Also presented are estimates of the graft polymer thickness that were made by using the tube dimensions and assuming a coating density of 1.0 g/cm³.

TABLE 1
Weight gain results and an estimation of graft thickness

| | Mass of | Mass of | | Estimated |
|---|---|---|---|---|
| Coating | uncoated | coated | Percent | coating |
| time | tubing | tubing | mass | thickness |
| (min) | (g) | (g) | increase | (μm)^{a} |
| 30 | 22.6420 | 22.9071 | 1.2 | 5.5 |
| 45 | 22.5548 | 23.0870 | 2.4 | 11 |
| 60 | 22.5359 | 23.2634 | 3.2 | 15 |

^{a}Tubing ID = 1.58 mm, OD = 3.18 mm, surface area = 1.49 cm²/cm tubing Coating density assumed to be 1.0 g/cm³.

### Example 12

### Preparation of a NHS-Activated Graft-Coat Layer Containing Acrylamide, Acrylic Acid and PEG-Methacrylate on Polyurethane Tubing Using no Precoat Layer

Application of Initiator Solution

A solution of radical initiator was prepared by dissolving lauryl peroxide, 4.01 g, tetrahydrofuran 50.09 g and acetone, 50.22 g. Five, four inch long pieces of polyurethane tubing (Tecothane TT-1095A, Thermedics), were dipped approximately 4.5 inches into and removed from the solution of initiator and allowed to dry at room temperature for one hour.

Application of Graft Polymer

A concentrated aqueous solution of sodium chloride was prepared by dissolving sodium chloride, 75 g in deionized water, 425 g. A reactive monomer solution was prepared by next dissolving acrylamide, 3.80 g, acrylic acid, 0.97 g, and S-20W poly(ethylene glycol) methacrylate, 2.43 g, in 240 g of the salt solution.

The five pieces of initiator coated polyurethane tubing were placed in a reactor vessel along with 124.2 g of the monomer solution described above. The reaction tube was degassed by twice repeated evacuation and flushing with nitrogen gas. The reactor system was equilibrated to atmospheric pressure of nitrogen and immersed in a preheated water bath (89° C.) for 45 minutes. The reactor was removed from the water bath and cooled. The coated slides were rinsed with copious amounts of water and allowed to dry at ambient conditions.

Activation of graft polymer carboxylic acid active sites with N-hydroxysuccinimide (NHS).

A solution was prepared by mixing 0.1 M MES buffer (2-[N-morpholino]ethanesulfonic acid, Sigma), 250 ml and 0.5 N aqueous sodium chloride, 250 ml. The pH of this solution was adjusted to 6 by addition of an aqueous solution of 0.8 N sodium hydroxide. pH measurement was performed using an Accumet pH Meter 50 (Fisher Scientific). The coated polyurethane tubing was immersed in 50 g of the above MES buffer solution. To this solution was added 1-ethyl-3-(3-dimethylaminopropyl) carbodiimide hydrochloride (EDC, Pierce), 0.020 g, and N-hydroxysuccinimide (Aldrich), 0.030 g. This was stirred for 15 minutes. The tubing was removed from this solution, immersed for 1 minute in deionized water, removed and dried under vacuum. Samples treated in this way were stored at room temperature under dry conditions until further use.

Determination of N-Hydroxysuccinimide Concentration on Polyurethane Surface

The NHS-GRAFT-COAT polyurethane tubing were cut into two, 2 cm lengths and placed into 1.8 ml of 0.05 N NaOH for 4 hours at room temperature. The sample was removed and 0.2 ml 10×PBS was added to the solution. The pH of the solution was adjusted to7.3 with 2 drops of aqueous 1N hydrochloric acid. The solution was scanned with a spectrophotometer. The absorbance value at 260 nm was measured and divided by the molar extinction coefficient (Example 8) to determine the total amount of NHS in solution. The concentration of NHS per linear centimeter was then determined to be 6.0 μg/cm by dividing the total amount of NHS by the length of the coated surface. The NHS concentration per unit area was calculated to be 10 μg/cm².

### Example 13

### Preparation of the Graft Polymer Layer Containing Acrylamide, Acrylic Acid, Poly(vinylpyrrolidone) and PEG-Methacrylate

Application of Primer

A solution of primer containing a radical initiator was prepared by mixing together a styrene-butadiene based adhesive (45% solid, Eclectic Products, Springfield Oreg.), 5.01 g, tetrahydrofuran, 22.5 g, toluene, 22.5 g, and lauryl peroxide, 0.124 g. The concentration of lauryl peroxide was 5.5 percent by weight of solids. The resulting solution was filtered through a 75 μm nylon mesh to remove particulate matter. Three standard sized (25 mm×75 mm×1.1 mm) microscope slides that had been previously cleaned by washing with tetrahydrofuran were dipped approximately 5 cm into and removed from the initiator/primer solution and dried at room temperature overnight.

Application of Graft Polymer

A concentrated aqueous solution of sodium chloride was prepared by dissolving sodium chloride, 330 g, and poly(vinylpyrrolidone) (K90, ISP), 4.4 g in deionized water, 1866 g. A reactive monomer solution was prepared by next dissolving acrylamide, 8.05 g, S-20W polyethylene glycol methacrylate, 4.62 g, acrylic acid, 0.0831 g in 337 g of the salt solution.

Three microscope slides were stacked together, placing a 2 cm piece of microscope slide between each slide to keep them separated. A portion of the monomer and salt solution prepared above, 124 g, and the slides were placed in a glass tube (6 cm×40 cm). The reaction tube was degassed by twice repeated evacuation and flushing with nitrogen gas. The reactor system was equilibrated to atmospheric pressure of nitrogen and immersed in a preheated water bath (89° C.) for 45 minutes. The reactor was removed from the water bath and cooled. The coated slides were rinsed with copious amounts of water and allowed to dry at ambient conditions.

NHS-Activation of Acrylic Carboxylic Acid Groups

A solution was prepared by mixing 0.1 M MES buffer (2-[N-morpholino]ethanesulfonic acid, Sigma), 100 ml and 0.5 N aqueous sodium chloride, 100 ml. The pH of this solution was adjusted to pH 6 by addition of an aqueous solution of 0.8 M sodium hydroxide. pH measurement was performed using an Accumet pH Meter 50 (Fisher Scientific). To a 140 ml aliquot of this solution was added 1-ethyl-3-(3-dimethylaminopropyl) carbodiimide hydrochloride (EDC, Pierce), 0.056 g, and N-hydroxysuccinimide (Aldrich), 0.084 g. Graft polymer-coated slides were immersed in the EDC/NHS solution and stirred for 15 minutes. Slides were removed from this solution, immersed for 1 minute in deionized water, and dried under vacuum. NHS-activated slides were stored at room temperature under dry conditions until further use.

Reaction of NHS-Activated Carboxylic Sites with Biocytin

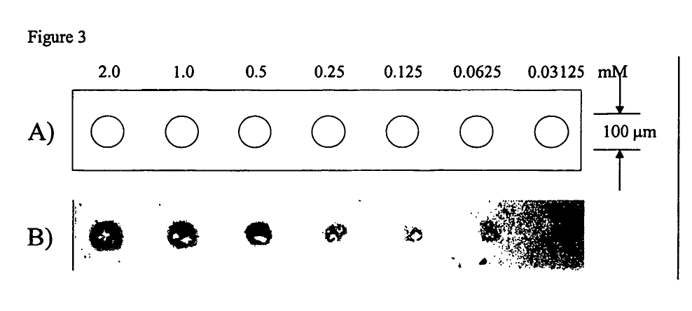

Seven serial dilutions of biocytin were prepared by dissolving biotin (Pierce) in the appropriate amount of phosphate buffered saline solution (PBS, 0.1 M sodium phosphate and 0.15 M sodium chloride, pH 7.2) to achieve biocytin concentrations of 2.0, 1.0, 0.5, 0.25, 0.125, and 0.0625, and 0.03125 mM. Spots 100 μm in diameter were Printed onto the NHS-activated slides using a Virtek Chip Writer Professional. Spots were allowed to dry for one hour at room temperature at a relative humidity of 45% to prevent the diffusion of biocytin. Arrays were subsequently washed three times for 5 minutes in 1×PBS. Room temperature Tris-glycine buffer, pH 7.4, (0.1 M Tris, 0.05 M glycine, 0.15 M NaCl pH 7.4) was added to the surface of the slide for 30 minutes with a change of fresh buffer at 15 minutes to block the array surface. Arrays were washed thoroughly with Tris-glycine buffer prior to staining with 0.025 mg/ml streptavidin-alkaline phosphatase in Tris-glycine buffer, pH 7.4. All arrays were washed 4 times for 5 minutes in 1×PBS, pH 7.4 following staining. Upon completion of washing the arrays were developed with NBT/BCIP substrate for 15 minutes in the dark followed by several distilled water rinses. Imaging was performed at a final magnification of 10× on an Olympus Provis microscope using a SPOT digital camera and MCID image analysis software. FIG. 3 shows an increase in color density that corresponds to an increase in printed biocytin concentration across the graft polymer surface.

### Example 14

### Preparation and Analysis of NH2-GRAFT-COAT on Slides Containing Two Concentrations of Initiator on the Slide Surface

Application of Primer Layer at a low 5% w/w Concentration

A solution of primer containing a radical initiator was prepared by mixing together a styrene-butadiene based adhesive (Eclectic Products, Springfield Oreg.), 5.45 g, tetrahydrofuran, 24.27 g, toluene, 24.25 g, and lauryl peroxide, 0.1280 g. The concentration of lauryl peroxide was 5 percent by weight of solids. Six standard sized (25 mm×75 mm×1.1 mm) glass microscope slides that had been previously cleaned by washing with tetrahydrofuran were dipped approximately 5 cm into and then removed from the initiator/primer solution and dried at room temperature overnight.

Application of Primer at a High 10% w/w Concentration.

A solution of primer containing a radical initiator was prepared by mixing together a styrene-butadiene based adhesive (Eclectic Products, Springfield Oreg.), 5.45 g, tetrahydrofuran, 24.17 g, toluene, 24.18 g, and lauryl peroxide, 0.2770 g. The concentration of lauryl peroxide was 10 percent by weight of solids. Six standard sized (25 mm×75 mm×1.1 mm) glass microscope slides that had been previously cleaned by washing with tetrahydrofuran were dipped approximately 5 cm into and then removed from the initiator/primer solution and dried at room temperature overnight.

Application of Graft Polymer Containing Acrylamide, Aminopropylacrylamide, and PEG-Methacrylate to the Low and High Initiator Primed Slide Samples

A concentrated aqueous solution of sodium chloride was prepared by dissolving sodium chloride, 555 g, and poly(vinylpyrrolidone) (K90 grade, BASF), 7.40 g, in deionized water, 3137.6 g. A reactive monomer solution was prepared by next dissolving acrylamide, 14.17 g, poly(ethylene glycol) methacrylate (S-20W grade, Laporte), 9.88 g, 3-aminopropylacrylamide hydrochloride, 6.34 g in 689.61 g of the salt solution.

The two sets of six glass microscope slides were stacked together by placing three 2 cm pieces of microscope slide between each slide to keep them separated, and then placed in a separate reactor vessels along with 180 g of the monomer solution. The reaction vessels were degassed by twice repeated evacuation and flushing with nitrogen gas. The reactor systems were equilibrated to atmospheric pressure of nitrogen and immersed in a preheated water bath (89° C.) for 30 minutes. The reactors were removed from the water bath and cooled. The coated slides were rinsed with copious amounts of water and allowed to dry at ambient conditions.

In this manner, slides were prepared having a graft polymer comprising polyethylene glycol structural modifier and alkylamine active groups attached to the graft polymer backbone.

Six more sets of glass slides were prepared using the same primer and monomer solutions, but the graft polymerization was carried out for 45, 60 and 120 minutes.

Analysis

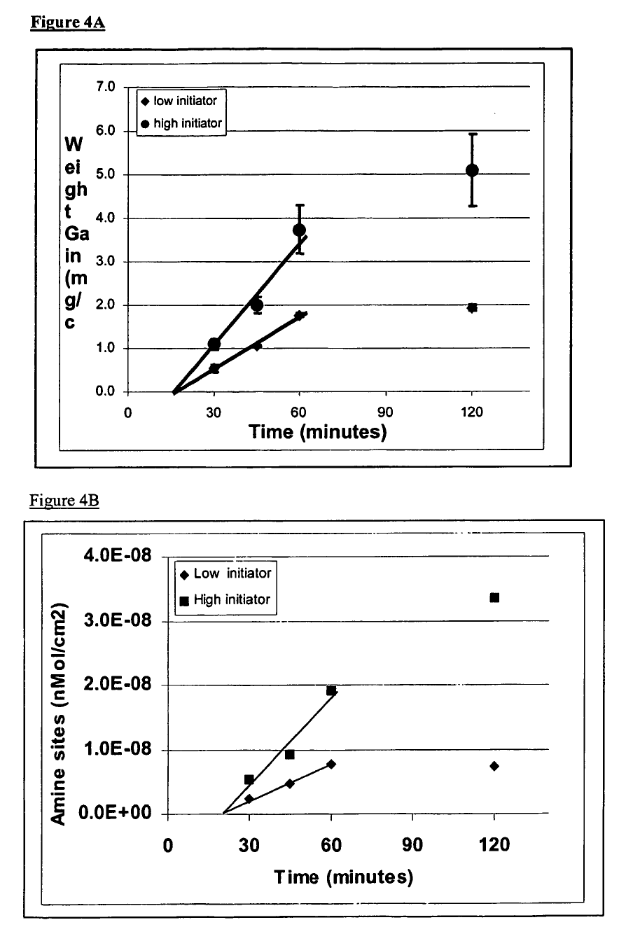

Graft coating thickness and density can be controlled by the reaction time and initiator concentration, respectively. Four types of analyses were performed at each time point; weight gain, determination of amount of NH2 groups, fluorescence isothiocyante (FITC) binding and depth measurement of FITC bound slides by confocal microscopy. FIG. 4A graphs the weight gain of both sets of slides, containing high and low initiator concentrations, versus time. A higher concentration of initiator lead to increased graft polymer coating. Weight gain corresponded to the amount of time in the reaction vessel and after a short lag phase (due to reaction vessel heating), was linear from 15 to 60 minutes. The higher initiator concentration resulted in more graft polymerization at each time point. The concentration of amino groups were quantified using a modified TNBS spectrophotometric assay. As graphed in FIG. 4B, the amount of amine groups also increased with time and was linear for the 15-60 minute time duration. Corresponding to the weight gain, the amount of amino groups was higher on slides primed with the higher concentration of initiator. Graft coated slides were further analyzed with FITC, an amine-specific fluorescent binding probe. Eighteen, 1:1 serial dilutions starting with 0.1 mg/ml of FITC in PBS, pH 7.5, were prepared. Samples (N=9 per dilution) were printed in a staggered pattern using a Virterk Chipwriter. After washing, the slides were scanned with a Virtek chip reader. FIG. 4C is a composite of images from each sample. A false color spectrum gradient was applied to each image (red indicating high fluorescence). Consistent with the weight gain and amount of incorporated amino groups, the fluorescence intensity increase with time and was greater with samples containing the higher initiator concentration.

These samples were also scanned with a confocal microscope and graft coating depth measurements were determined for each sample. FIG. 4D graphs the graft coating thickness as a function of time for both low and high initiator primed samples. The thickness increased with time indicating the elongation of the graft polymer chains. The data from slides primed with low and high initiator were superimposable, indicating that the parallel increase in thickness was not dependent on the initiator concentration. Thus, the increase in polymer weight and amino groups is due to an increase in graft polymer chain surface density and not coating thickness. Therefore, the initiator concentration dictates the graft polymer surface density and the reaction time, the polymer chain length.

### Example 15

### Preparation of the Graft Polymer Layers Containing N,N Dimethylacrylamide Backbone, with and without Carboxylic Acid Active Sites

The carboxylic acid derivatives include; acrylic acid short side chain active site, beta-carboxyethyl acrylate medium side chain and beta-acryloyl oxyethyl hydrogen succinate long side chain active site. Also, an acrylamide backbone version of the beta-acryloyl oxyethyl hydrogen succinate was prepared.

Application of Primer

A solution of primer containing a radical initiator was prepared by mixing together a styrene-butadiene based adhesive (45% solid, Eclectic Products, Springfield Oreg.), 15.03 g, tetrahydrofuran, 67.32 g, toluene, 67.42 g, and lauryl peroxide, 0.36 g. The concentration of lauryl peroxide was 5 percent by weight of solids. Twenty-five standard sized (25 mm×75 mm×1.1 mm) microscope slides that had been previously cleaned by washing with tetrahydrofuran were dipped approximately 5 cm into and removed from the initiator/primer solution and dried at room temperature overnight.

Preparation of Monomer and Salt Solution

A concentrated aqueous solution of sodium chloride was prepared by dissolving sodium chloride, 555 g, and poly(vinylpyrrolidone) (K90 grade, BASF) 7.4 g, in deionized water, 3137.6 g.
15A Application of graft polymer N,N dimethylacrylamide backbone only control

A reactive monomer solution was prepared by dissolving N,N dimethylacrylamide, 9.85 g, in 290.26 g of the salt solution.
15B Application of graft polymer with a short side chain carboxylic acid and N,N dimethylacrylamide backbone

A reactive monomer solution was prepared by dissolving N,N dimethylacrylamide, 9.37 g, and acrylic acid, 0.35 g, in 290.34 g of the salt solution.
15C Application of graft polymer with a medium length side chain carboxylic acid and N,N dimethylacrylamide backbone

A reactive monomer solution was prepared by dissolving N,N dimethylacrylamide, 9.33 g, and beta-carboxyethyl acrylate, 0.71 g, in 289.97 g of the salt solution
15D Application of graft polymer with a long side chain carboxylic acid and N,N dimethylacrylamide backbone

A reactive monomer solution was prepared by dissolving N,N dimethylacrylamide, 9.34 g, and beta-acryloyl oxyethyl hydrogen succinate, 1.08 g, in 289.61 g of the salt solution.
15E Application of graft polymer with a long side chain carboxylic acid and an acrylamide backbone

A reactive monomer solution was prepared by dissolving acrylamide, 6.71 g, and beta-acryloyl oxyethyl hydrogen succinate, 1.09 g, in 292.26 g of the salt solution.

Application of Graft-Coat to Primed Glass Slide Surfaces

Five primer coated microscope slides were stacked together, placing three 2 cm piece of microscope slide between each slide to keep them separated. A 150 g portion of the monomer salt solutions prepared above and the slides were placed in a glass tube (6 cm×40 cm). The reaction tube was degassed by twice repeated evacuation and flushing with nitrogen gas. The reactor system was equilibrated to atmospheric pressure of nitrogen and immersed in a preheated water bath (89° C.) for 60 minutes. The reactor was removed from the water bath and cooled. The coated slides were rinsed with copious amounts of water and allowed to dry at ambient conditions.

Activation of Graft Polymer Carboxylic Acid Active Sites with N-Hydroxysuccinimide (NHS)

A solution was prepared by mixing 0.1 M MES buffer (2-[N-morpholino]ethanesulfonic acid, Sigma), 500 ml and 0.5 N aqueous sodium chloride, 500 ml. The pH of this solution was adjusted to pH 6 by addition of an aqueous solution of 30% sodium hydroxide. Each slide was immersed in 20 ml of this solution for 30 minutes. One-ethyl-3-(3-dimethylaminopropyl) carbodiimide hydrochloride (EDC, Pierce), 0.047 g, and N-hydroxysuccinimide (Aldrich), 0.020 g per slide were then added to the buffer solution and slide with stirring. Primer-graft coated slides from Example 15 were held in the buffer, EDC/NHS solution and stirred for 120 minutes. Slides were removed from this solution, washed in deionized water three times, and dried under vacuum. NHS-activated slides were stored at room temperature under dry conditions until further use.

Analysis

Table 2 lists the mole percents of the respective monomers that were in the reactive monomer salt solutions. The measured NHS concentrations are the moles of NHS bound per slide surface area. It is known in the art that monomers react at different rates. Thus, it is not surprising that different NHS concentrations were found even though each graft polymerization started out with a 5 mole % carboxylic acid monomer. One skilled in the art can compensate for these different monomer reaction rates. For example, by changing the starting mole percents of the reactive monomers or changing the reaction conditions, such as time, temperature, salt concentration, initiator, etc. to arrive at a desired final graft polymer matrix structure.

TABLE 2

| | | Mole % | Mole % | NHS |
|---|---|---|---|---|
| | | Back- | Carboxylic | Concentration |
| | Backbone | bone | Acid | moles/cm² |
| 15A | N,N dimethylacrylamide | 100% | 0% | NA |
| 15B | N,N dimethylacrylamide | 95% | 5% | 3.10E−09 |
| 15C | N,N dimethylacrylamide | 95% | 5% | 1.20E−08 |
| 15D | N,N dimethylacrylamide | 95% | 5% | 8.00E−09 |
| 15E | acrylamide | 95% | 5% | 1.50E−08 |

Macroanalysis

Working solutions of biotin and biocytin were prepared at 1×10-6 mol/ml. Nine serial dilutions (1:1) in PBS pH 7.5 were made. One microliter of each dilution was hand pipetted onto the graft coat slide surfaces.

Microanalysis

Working solutions of biotin and biocytin were prepared at 1×10-7 mol/ml. Twenty-four serial dilutions (1:1) in PBS pH 7.5 were made. Ten μl of the biotin or biocytin dilutions were pipetted into wells of a 384 microtiter plate. The samples were printed on slides containing the various graft coat surfaces with a Virtek Chipwriter at room temperature and 55% relative humidity.

For both the macroanalysis and microanalysis, after printing, slides were washed and blocked three times for 15 minutes with 0.1 M tris/0.05M glycine, pH 7.4 with shaking. The slides were then dried by centrifugation. One hundred μl of a streptavidin conjugated to Cy3 fluorochrome (SA-Cy3, 0.025 mg/ml in PBS) solution was added to the sample surfaces followed by placing 24×60 mm coverslips on the slides without introducing air bubbles. Incubation was at room temperature in a humidity chamber for 30 minutes. Samples were protected from light. The coverslips were removed by dunking in PBS, followed by washing four times in PBS with a final water rinse prior to drying by centrifugation. The slides were then scanned with a Virtek chipreader. A false color spectrum was introduced to the digitized images with Adobe Photoshop software.

NH2-Oligos and control oligos and cDNA were printed at a concentration of 50 μM in 15 mM Sodium Citrate/150 mM Sodium Chloride, pH 7.5. Ten μl of oligo and cDNA samples were pipetted into a 384 well plate and printed at 55% humidity at room temperature with a Virtek chipwriter using smp3b Array It stealth pins. Slides were then washed and blocked three times for 15 minutes with 0.1 M tris/0.05M glycine, pH 7.4 with shaking. Target hybridization was done with Spotcheck (Genpak Inc. Stony Brook N.Y.) a random Cy3 labeled oligo. Fifty μl of Spotcheck was denatured at 95 C for 5 minutes then cooled immediately on ice. The mixture was pipetted onto the array surface and covered with a 24×50 mm coverslip, avoiding air bubbles. The slide was placed in a humidity chamber at room temperature and incubated for 30 minutes in the dark. The slides were washed at room temperature in a Wheaton stain dish with SSC with 0.1% SDS for 10 min. followed by multiple washes in SSC. Slides were dried by centrifugation at 1000 rpm for 5 minutes and stored in a dessicator prior to scanning with a Virtek Chipreader.

Imaging Results

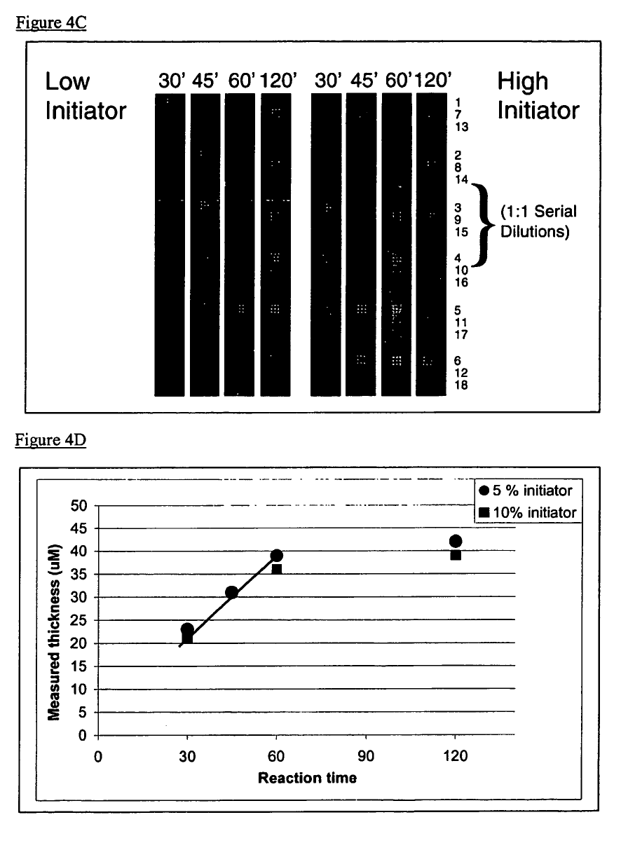

FIG. 5A is a composite image of NHS-graft coat slides (15A, 15B and 15D) macroprinted with serial dilutions of biocytin (left) and biotin (right). The control slide (15A) did not have binding of either biocytin or biotin. 15B (short spacer arm) exhibited specific binding with biocytin and no binding with control biotin. 15D (long spacer arm) had a higher absolute binding with biocytin and no binding with control biotin. FIG. 5B is a graphic representation of the same experiment where pixel density per spot is plotted against the concentration of biocytin in the printing solution. The NHS-graft coat sample with the short spacer arm (15B) bound biocytin but less efficiently compared to the NHS-graft coat containing a long spacer arm (15D) as indicated by the saturation of 15D at much higher SA-Cy3 fluorescence intensity levels.

FIG. 5C is a composite microarray image where biocytin was printed onto NHS-graft-coat slides with different length spacer arms (15D-long, 15C-medium and 15B-short). The SA-Cy3 binding fluorescence intensity was high for all samples (15D-highest) compared to low binding seen with commercially available NHS-surface treated slides and control graft coat slides containing no NHS functional groups.

Good results were also achieved with a graft polymer containing an acrylamide backbone (15E) with the long spacer arm NHS active sites. But background activity was generally higher and less uniform. The acrylamide backbone also provides a more hydrophilic probe and target binding environment.

The printed pattern NH2-oligos, control oligos and corresponding cDNA were distributed through out a 10 column by 12 row subgrid. Column 1 consisted of only buffer and column 2 cDNA, both showed no fluorescent signal. Columns 7-10 contained NH2-oligos which had the highest signal intensity as evident by all spots with a false color of violet or higher. Control oligos demonstrated minimal fluorescent signal as demonstrated with spots in columns 4-6 rows 1-3. Column 4-6, rows 7-12 contained NH2-oligos which also had an increased signal compared to the control oligos.

### Example 16

### Preparation of a Graft Polymer Layer Containing Acrylamide, Aminopropylacrylamide, and PEG-Methacrylate with a Silane Monomer Primer Layer

Application of Silane Monomer Primer

Twelve standard sized (25 mm×75 mm×1.1 mm) glass microscope slides were cleaned by immersing in 2.5% w/w NaOH solution for ten minutes, then rinsing in deionized water and wiping dry. A silane monomer solution was prepared by mixing together 186.2 g of ethanol, 9.8 g of deionized water and 4.0 g of methacryloxypropyl-trimethoxy-silane. The cleaned glass slides were held in the silane solution for five minutes, rinsed with ethanol and dried at 110 C for 5 minutes.

A solvent solution containing a radical initiator was prepared by mixing together 24.75 g of tetrahydrofuran, 24.75 g of toluene, and 0.50 g of lauryl peroxide. The concentration of lauryl peroxide was 1 percent by weight of solution. Six of the silane treated slides were dip coated with the solvent initiator solution sample 16A. Six silane treated slides were not coated with the initiator solution, sample 16B.

Application of Graft Polymer

A concentrated aqueous solution of sodium chloride was prepared by dissolving sodium chloride, 555 g, and poly(vinylpyrrolidone) (K90 grade, BASF), 7.40 g, in deionized water, 3137.6 g. A reactive monomer solution was prepared by next dissolving acrylamide, 5.92 g, poly(ethylene glycol) methacrylate (S20W Laporte), 4.14 g, 3-aminopropylacrylamide hydrochloride, 2.49 g in 287.54 g of the salt solution.

The six 16A and six 16B treated glass microscope slides were stacked together separately, placing three 2 cm pieces of microscope slide between each slide to keep them apart, and then placed in separate reactor vessels along with 150 g of the monomer salt solution. The reaction tubes were degassed by twice repeated evacuation and flushing with nitrogen gas. The reactor systems were equilibrated to atmospheric pressure of nitrogen and immersed in a preheated water bath (89° C.) for 30 minutes. The reactors were removed from the water bath after 60 minutes and cooled. The coated slides were rinsed with copious amounts of water and allowed to dry at ambient conditions.

In this manner, slides were prepared having a graft polymer matrix comprising a polyethylene glycol structural modifier and alkylamine active groups with an acrylamide backbone attached to the glass surface by the silane methacrylate monomer.

The primary amine concentration was measured by the modified TNBS method described in example 14.

Sample 16A silane methacrylate monomer plus initiator 1.4×10⁻⁰⁸ moles/cm2

Sample 16B silane methacrylate monomer no initiator 2.3×10⁻¹⁰ moles/cm2

Sample 16A had two orders of magnitude more primary amines present indicating the growth of graft copolymer chains from the initiated silane monomer surface.

### Example 17

### Anti-Infective Graft Polymer Matrices on Helix Medical Tubing

The same methods and procedures were used as in example 11 but the initiator and monomer solutions were modified as follows to produce the samples after 60 minutes of graft coat reaction time.

TABLE 3

| | Initiator | Acrylamide | Acrylic Acid | Zone |
|---|---|---|---|---|
| Sample | Concentration | Mole Percent | Mole Percent | Size (mm) |
| 17A | 0.5% w/w | 95% | 5% | 0 |
| 17B | 1% w/w | 95% | 5% | 7.2 |
| 17C | 2% w/w | 95% | 5% | 7.8 |
| 17D | 2% w/w | 85% | 15% | 8.4 |

Each sample was immersed in a 0.5 molar silver nitrate solution over night. The samples were washed in deionized water ten times and then dried at room temperature.

They were incubated for 24 hours at 37 C on standard agar S. aureus and evaluated by zone of inhibition. A clear zone around the sample indicates anti-infective activity. The results set forth in Table 3 show increasing zone sizes with increasing amounts of acrylic acid on the silicone tubing, indicating silver attachment to the graft polymer matrix through the acrylic acid sites.

### Example 18

### Preparation of a Graft Polymer Layer Containing Acrylamide, Aminopropylacrylamide, and PEG-Methacrylate with Initiator Incorporated into the Primer at Low 5% w/w Concentration Followed by Terminal Sulfhydryl Group Activation of the Primary Amine Active Sites

Ten primary amine active site slides were prepared in the same manner as in Example 14, low 5% initiator and 60 minute time point. They were analyzed for amino group concentration by a modified TNBS assay. The NH₂ concentration was 3.1×10⁻⁰⁹ Moles/cm². These slides were each treated with 5 ml of 8 mM Traut's reagent (2-iminothiolane) in PBS/1 mM EDTA pH 8 for 2 hours at room temperature. The SH-graft coat slides were washed in PBS pH 8, and treated with 10 mM dithiothreitol (DTT) in PBS/EDTA for 15 minutes. Slides were washed exhaustively in PBS followed by incubation with 10 mM Ellman's reagent (5,5′Dithio-bis-[nitrobenzoic Acid]) in PBS/EDTA for 60 minutes. To determine sulfhydryl concentration, slides were washed with PBS followed by the addition of 5 ml of 10 mM DTT per slide to cleave bound reagent. The absorbance of the DTT solutions at 412 nm (E_{1M}=14100) was used to indirectly determine the sulfhydryl concentration on the slides.

| Slide ID | [SH] mol/cm² |
|---|---|
| GO020502A | 1.40 × 10⁻⁹ |
| GO020502B | 1.78 × 10⁻⁹ |
| GO020502C | 1.52 × 10⁻⁹ |
| Average | 1.57 × 10⁻⁹ |

SH-graft coat slides were printed with acryldite-modified oligo, nonmodified oligos and controls, under both high (40 micromolar) and low (8 micromolar) concentrations. Each sample was printed with 5 replicates per row with each row containing a different sample. Hybridization was then performed with oligo-Cy3.

Acrydite is a molecular chimera containing an acrylic group and phosphoramidite that can be used as a platform for oligo construction (via the phosphoramidite part) and as a vehicle to attach oligos to surfaces (via the acrylic portion). What makes this particular molecule so useful is that it attaches to surfaces in a single step—and a number of different surfaces are possible—forms a bond that is resistant to high temperatures, and leaves the attached oligo accessible for hybridization assays that are both rapid and efficient.

Results

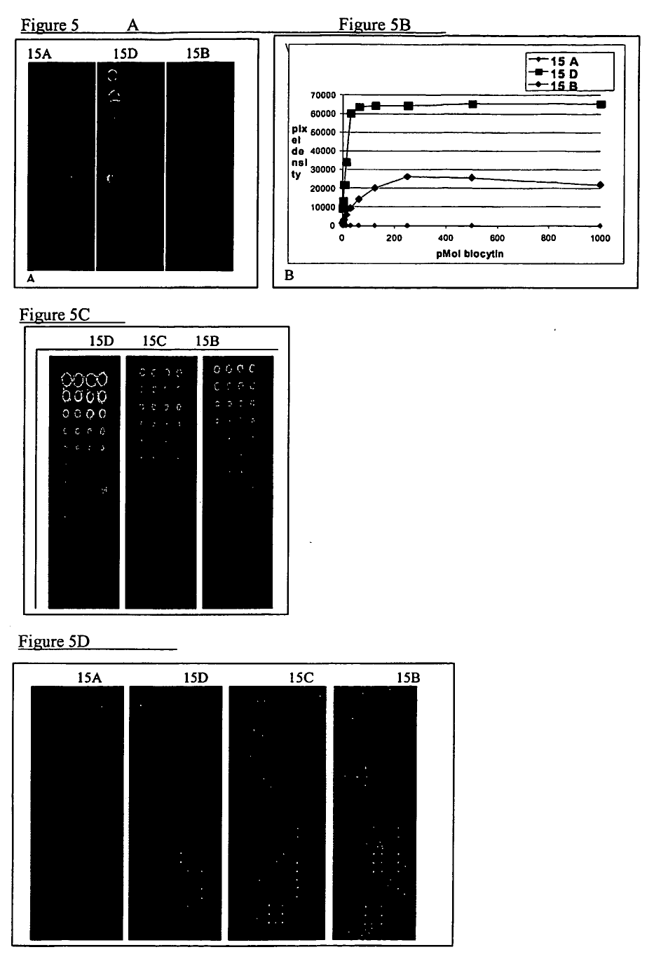

FIG. 6 is an image of the scanned SH-graft polymer microarray slide printed with acrydite-modified oligos, control oligos and other reagents. Good target-Cy3 binding was evident with the printed acryldite-oligos (row 3). All other controls had low binding (row-1, food dye; row-2 and 4, buffer; row 5, control oligo).

### Example 19

### Preparation of a Graft Polymer Layer Containing Acrylamide, PEG-Methacrylate and Aminopropylacrylamide Varying in Concentration in the Monomer Solution from 2.5 to 15 Mole Percent

Application of Primer

A solution of primer containing a radical initiator was prepared by mixing together a styrene-butadiene based adhesive (Eclectic Products, Springfield Oreg.), 5.45 g, tetrahydrofuran, 24.17 g, toluene, 24.18 g, and lauryl peroxide, 0.2770 g. The concentration of lauryl peroxide was 10 percent by weight of solids. Six standard sized (25 mm×75 mm×1.1 mm) glass microscope slides that had been previously cleaned by washing with tetrahydrofuran were dipped approximately 5 cm into and then removed from the initiator/primer solution and dried at room temperature overnight.

Application of Graft Polymer to Initiator Primed Slide Samples

A concentrated aqueous solution of sodium chloride was prepared by dissolving sodium chloride, 555 g, and poly(vinylpyrrolidone) (K90 grade, BASF), 7.40 g, in deionized water, 3137.6 g. A reactive monomer solution was prepared by next dissolving aminopropylacrylamide hydrochloride at increasing mole percentages of 2.5, 5, 10 and 15. In these mixtures the amount of acrylamide monomer was 96.5, 94.0, 89.0 and 84.0 percent, respectively. All solutions contained 1 mole percent poly(ethylene glycol) methacrylate monomer.

The two sets of six glass microscope slides were stacked together by placing three 2 cm pieces of microscope slide between each slide to keep them separated, and then placed in a separate reactor vessels along with 180 g of the monomer solution. The reaction vessels were degassed by twice repeated evacuation and flushing with nitrogen gas. The reactor systems were equilibrated to atmospheric pressure of nitrogen and immersed in a preheated water bath (89° C.) for 60 minutes. The reactors were removed from the water bath and cooled. The coated slides were rinsed with copious amounts of water and allowed to dry at ambient conditions. In this manner, slides were prepared having a graft polymer solution comprising polyethylene glycol structural modifier and alkylamine active groups present in increasing amounts.

Analysis

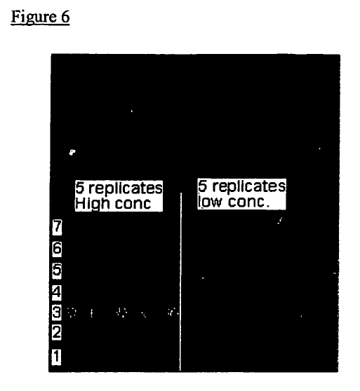

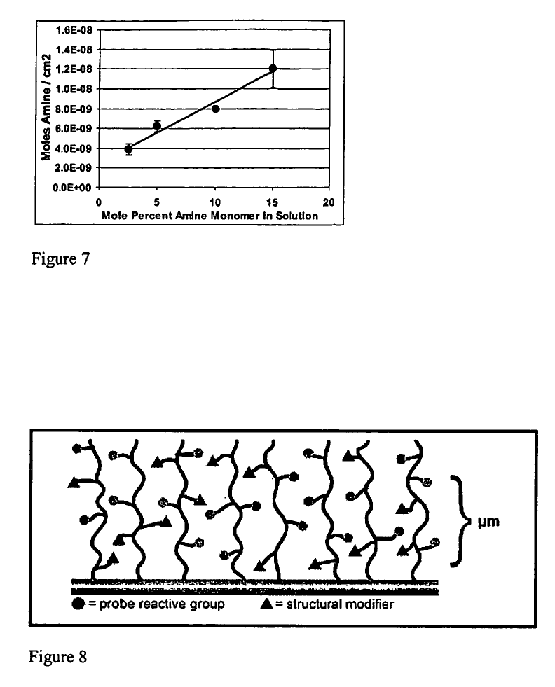

To demonstrate the control of the number functional sites per polymer chain, monomers solutions containing increasing mole percents of primary amine were prepared. The resultant amount of primary amine monomer incorporated into GRAFT-COAT was determined spectrophotometrically by using a modified TNBS assay (FIG. 7). The amount of bound amino groups incorporated into the graft polymer increased linearly corresponding to the increase in concentration of the aminopropylacrylamide hydrochloride monomer in the reaction mixture.

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

1. A three-dimensional, non-crosslinked, linear or branched graft polymer matrix comprising one or more active chemical moieties having inherent specificity for binding to a chemical, biochemical, or biological probe, wherein said moieties are permanently attached to and distributed throughout the graft polymer matrix.

2. The matrix of claim 1 additionally comprising at least one probe having selective affinity for a target.

3. The matrix of claim 1 wherein the matrix is comprised of linear or branched graft polymer molecules of controlled length and density.

4. The matrix of claim 1 wherein the active chemical moieties are attached as side chains to the backbone of the graft polymer chain.

5. The matrix of claim 1 wherein the probe is a nucleic acid or protein.

6. The matrix of claim 1 wherein the probe is a toxin, pathogen or pharmaceutical agent.

7. The matrix of one of claim 2, wherein the target is selected from the group consisting of viruses, bacteria, fungi, parasites, and molecules or molecular fragments of DNA, RNA, proteins, carbohydrates and lipids.

8. The matrix of claim 1 additionally comprising at least one structural modifier.

9. The matrix of claim 1 wherein the active chemical moieties are selected from the group consisting of amines, carboxylic acids, epoxides, aldehydes, sulfhydryls, haloacetamides and carboxylic acid succinimidyl esters.

10. The matrix of claim 9 wherein the active chemical moiety is a N-hydroxysuccinimidyl ester of a carboxylic acid.

11. The matrix of claim 1 that contains a plurality of different chemical moieties.

12. The matrix of claim 1 wherein the chemical moieties are further modified to alter their reactivity.

13. The matrix of claim 12 wherein the chemical moieties are side-chain chemical moieties, and are further modified with spacer groups to further enhance their reactivity.

14. An article comprising the matrix of claim 1.

15. (canceled)

16. (canceled)

17. (canceled)

18. (canceled)

19. An article having on its surface a coating comprising a three-dimensional, non-crosslinked, linear or branched graft polymer matrix, bonded to the article, comprising one or more chemical moieties that are permanently attached to and distributed throughout the graft polymer matrix wherein said chemical moieties impart specific functional utility to the article.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. An article having on its surface a multilayer coating comprising:
a) an adhesive layer comprising a polymer or polymer mixture, bound to the article surface; and
b) a three-dimensional, non-crosslinked, linear or branched graft polymer matrix, bonded to the polymer layer, comprising one or more chemical moieties that are permanently attached to and distributed throughout the graft polymer matrix, wherein said chemical moieties impart specific functional utility to the article.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A method for applying a three-dimensional, linear or branched graft polymer matrix to an article with a controlled graft polymer surface density, said method comprising the steps of:
a) applying to said surface a solution comprising:
i) an organic solvent or mixture of organic solvents,
i) a polymer or mixture of polymers which is soluble in said solvent or solvent mixture and insoluble in water, and
iii) a radical initiator that is soluble in said solvent or solvent mixture, said initiator being capable of generating reactive radical sites on the polymer-coated surface and initiating a graft polymerization reaction on said surface by generating reactive radical sites thereon;
b) removing the solvents to leave upon the polymer-coated surface a coating of initiator dissolved in polymer;
c) immersing the polymer coated surface in a medium comprising one or more monomers in solution that are capable of reacting with the reactive sites created by surface bound initiator to form a polymer chain grafted onto said polymer-coated surface;
d) initiating a graft polymerization reaction on said polymer-coated surface by generating reactive radical sites thereon;
e) graft polymerizing onto the polymer-coated surface the reactive monomers from the medium by forming covalent bonds between monomer molecules and the polymer-coated surface at reactive radical sites on the polymer-coated surface;
to obtain a coated article.

57. (canceled).

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. A method for applying a three-dimensional, linear or branched graft polymer matrix with a controlled graft polymer surface density to an article, said method comprising the steps of:
a) applying to said surface a solution comprising:
i) an organic solvent or mixture of organic solvents and
ii) a polymer or mixture of polymers which is soluble in said solvent or solvent mixture and insoluble in water;
b) removing the solvents to obtain a polymer-coated surface;
c) exposing said polymer-coated surface to an initiator capable of generating reactive radical sites on the polymer coated surface and initiating a graft polymerization reaction on said surface by generating reactive radical sites thereon;
d) immersing the polymer-coated surface in a medium comprising one or more monomers in solution that are capable of reacting with the reactive sites created by surface bound initiator to form a polymer chain grafted onto said surface;
e) initiating a graft polymerization reaction on said coated surface by generating reactive radical sites thereon;
f) graft polymerizing onto the coated surface the reactive monomers from the medium by forming covalent bonds between monomer molecules and the surface at reactive radical sites on the polymer coated surface;
to obtain an article with a coated surface having a controlled graft polymer surface density.

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. A method for applying a three-dimensional, non-crosslinked, linear or branched graft polymer matrix with a controlled graft polymer surface density to an article, said method comprising the steps of:
a) applying to said surface a solution comprising:
i) an organic solvent or mixture of organic solvents with water, and
ii) a silane monomer or mixture of silane monomers or nonreactive silanes which is soluble in said solvent or solvent water mixture;
b) removing the solvent to obtain a coated surface with silane attached monomers or other silane attached non-reactive groups upon the surface;
c) exposing said coated surface to an initiator capable of generating reactive radicals on the surface and initiating a graft polymerization reaction on said surface by generating reactive radical sites thereon;
d) immersing the coated surface in a medium comprising one or more monomers in solution that are capable of reacting with the reactive radical sites created by surface bound initiator to form a polymer chain grafted onto said surface; and
e) initiating a graft polymerization reaction on said coated surface by generating reactive radical sites thereon;
f) graft polymerizing onto the coated surface the reactive monomers from the medium by forming covalent bonds between monomer molecules and the surface at reactive radical sites on the coated surface
to obtain an article with a three-dimensional, non-crosslinked, linear or lightly branched graft polymer matrix with a controlled graft polymer surface density.

103. (canceled)

104. (canceled)

105. (canceled)

106. (canceled)

107. (canceled)

108. (canceled)

109. (canceled)

110. (canceled)

111. (canceled).

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. A method for applying a three-dimensional, non-crosslinked, linear or lightly branched graft polymer matrix with a controlled graft polymer surface density to an article, said method comprising the steps of:
a) applying to said surface a solution comprising:
i) an organic solvent or mixture of organic solvents with water and
ii) a silane with an attached initiator a or mixture of nonreactive silanes with attached initiators or silanes which is soluble in said solvent or solvent water mixture;
b) removing the solvents to leave a coated surface with silane attached initiators or other silane attached non-reactive groups upon the surface,
c) immersing the coated surface in a medium comprising one or more monomers in solution that are capable of reacting with the reactive sites created by surface bound initiator to form a polymer chain grafted onto said surface;
d) initiating a graft polymerization reaction on said coated surface by generating reactive radical sites thereon; and
e) graft polymerizing onto the coated surface the reactive monomers from the medium by forming covalent bonds between monomer molecules and the surface at reactive radical sites on the coated surface;
to obtain an article having a three-dimensional, non-crosslinked, linear or lightly branched graft polymer matrix with a controlled graft polymer surface density.

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. A method of making a microarray comprising the steps of
a) applying the matrix of claim 1 to a surface, wherein said matrix contains chemical moieties that react with, or can be modified to react with a biomolecule;
b) providing a solution of a probe biomolecule;
c) applying said solution to the matrix under conditions such that said probe biomolecules become bound to said chemical moieties.

145. (canceled)

146. (canceled)

147. A microarray made by the method of claim 144.

148. A biochip comprising the microarray of claim 147.

149. A method of detecting a target biomolecule, comprising contacting the biochip of claim 148 with a test sample in which the target biomolecule may be present.
